SEQUENCE LISTING (18 sequences)

SEQ ID NO: 1 - protein, 9 aa, Synthetic construct
ILKNLSRSR

SEQ ID NO: 2 - protein, 4 aa, Synthetic construct
VVAA

SEQ ID NO: 3 - protein, 6 aa, Synthetic construct
VVVAAA

SEQ ID NO: 4 - protein, 4 aa, Synthetic construct
EEEE

SEQ ID NO: 5 - protein, 4 aa, Synthetic construct
AAVV

SEQ ID NO: 6 - protein, 6 aa, Synthetic construct
AAAVVV

SEQ ID NO: 7 - protein, 5 aa, Synthetic construct
VVAAA

SEQ ID NO: 8 - protein, 5 aa, Synthetic construct
VVVAA

SEQ ID NO: 9 - protein, 4 aa, Synthetic construct
KKKK

SEQ ID NO: 10 - protein, 6 aa, Synthetic construct
VVAAEE

SEQ ID NO: 11 - protein, 4 aa, Synthetic construct
AAGG

SEQ ID NO: 12 - protein, 6 aa, Synthetic construct
AAAGGG

SEQ ID NO: 13 - protein, 4 aa, Synthetic construct
GGGG

SEQ ID NO: 14 - protein, 5 aa, Synthetic construct
VEVAA

SEQ ID NO: 15 - protein, 9 aa, Synthetic construct
LRNKSRILS

SEQ ID NO: 16 - protein, 7 aa, Synthetic construct
VVAAEEK

SEQ ID NO: 17 - protein, 15 aa, Synthetic construct
VVAAEEILKNLSRSR

SEQ ID NO: 18 - protein, 15 aa, Synthetic construct
VVAAEELRNKSRILS

(19) United States
(12) Patent Application Publication
(10) Pub. No.: US 2026/0192016 A1
(43) Pub. Date: Jul. 9, 2026

(54) GDNF MIMETIC PEPTIDE AMPHIPHILES

(71) Applicant: Northwestern University, Evanston, IL (US)

(72) Inventors: Samuel Isaac Stupp, Evanston, IL (US); Oscar Alejandro Carballo Molina, Evanston, IL (US)

(21) Appl. No.: 19/382,566

(22) Filed: Nov. 7, 2025

Related U.S. Application Data
(60) Provisional application No. 63/718,061, filed on Nov. 8, 2024.

(51) Int. Cl. A61L 27/22; A61L 27/38; C07K 14/475; C08L 89/00; C12N 5/00

(52) U.S. Cl. CPC A61L 27/227; A61L 27/38; C07K 14/4756; C08L 89/00; C12N 5/0068; A61L 2400/12; A61L 2430/32; C12N 2533/50

(57) ABSTRACT

Provided herein are peptide amphiphiles (PAs) and supramolecular PA nanostructures that mimic glial derived neurotrophic factor (GDNF), a growth factor that induces neuronal survival, maturation, and increased electrical activity. In particular, injectable biomaterials comprising GDNF mimetic PAs are provided, as well as methods of using GDNF mimetic PAs for the treatment or prevention of neurological injuries, diseases, and disorders, including concurrently with cell replacement therapy.

### STATEMENT OF RELATED APPLICATIONS

This application claims priority to and the benefit of U.S. Provisional Patent Application No. 63/718,061, filed Nov. 8, 2024, the entire contents of which are incorporated herein by reference for all purposes.

### STATEMENT OF GOVERNMENT SUPPORT

This invention was made with government support under grant number DGE1842165 awarded by the National Science Foundation. The government has certain rights in the invention.

### SEQUENCE LISTING

The text of the computer readable sequence listing filed herewith, titled “NWEST_43770_202_SequenceListing.xml”, created Mar. 25, 2026, having a file size of 16,528 bytes, is hereby incorporated by reference in its entirety.

### FIELD

Provided herein are peptide amphiphiles (PAs) that mimic glial cell derived neurotrophic factor (GDNF), and uses thereof in cell/organoid culture and methods of treating neurological disease.

### BACKGROUND

Parkinson's disease (PD) is recognized as the second most common neurodegenerative disease. The pathological hallmark of this disease is the loss of dopaminergic neurons in the substantia nigra. Given the relatively small number of neurons involved and their restricted distribution in the brain, cell replacement therapy has been explored as a therapeutic strategy, particularly for late-stage PD patients. However, outcomes of clinical trials with transplantation of dopaminergic neurons have been inconsistent. This discrepancy could be attributed to several factors, particularly to the poor the survival of transplanted neurons. In animal studies, the percentage of dopaminergic neurons that survive transplantation is typically less than 15%. Glial cell derived neurotrophic factor (GDNF) enhances the survival of transplanted dopaminergic neurons, but the ability of GDNF to promote survival is also limited by its degradation and several side effects, including off-state dyskinesias. In addition, there is high degree of inconsistency in using GDNF as a treatment to diminish the symptoms in PD patients. As such, there remains a need for clinically effective methods of improving survival of neurons, in particular for treating Parkinson's Disease.

### SUMMARY

In some aspects, provided herein are GDNF mimetic peptide amphiphiles. In some embodiments, provided herein is a GDNF mimetic peptide amphiphile comprising a hydrophobic tail, a structural peptide segment, a charged peptide segment, and a glial derived neurotrophic factor (GDNF) mimetic peptide. In some embodiments, the GDNF mimetic peptide comprises a sequence having at least 75% identity with ILKNLSRSR (SEQ ID NO: 1). In some embodiments, the GDNF mimetic peptide comprises a sequence having at least 88% identity with SEQ ID NO: 1. In some embodiments, the GDNF mimetic peptide comprises SEQ ID NO: 1.

In some embodiments, the hydrophobic tail comprises an 8-24 carbon alkyl chain (C₈₋₂₄). In some embodiments, the hydrophobic tail comprises a 16 carbon alkyl chain (C₁₆). In some embodiments, the structural peptide segment has propensity for forming β-sheet conformations. In some embodiments, the structural peptide segment comprises 2 to 8 non-polar residues. For example, in some embodiments the structural peptide segment comprises VVAA (SEQ ID NO: 2).

The charged peptide segment may comprise an acidic, basic, or zwitterionic peptide segment. In some embodiments, the charged peptide segment comprises EE, EEE, EEEE (SEQ ID NO: 4), KK, KKK, or KKKK (SEQ ID NO: 9). In some embodiments, the GDNF mimetic peptide is attached to the charged peptide segment by a linker.

In some embodiments, the peptide amphiphile comprises C₈₋₂₄-VVAAEEILKNLSRSR (SEQ ID NO: 17).

In some aspects, provided herein is a nanofiber comprising a GDNF mimetic peptide amphiphile provided herein. In some embodiments, the nanofiber comprises one or more filler peptide amphiphiles, wherein the filler peptide amphiphiles comprise a hydrophobic tail, a structural peptide segment, and a charged peptide segment, and wherein the filler peptide amphiphiles do not comprise the GDNF mimetic peptide. In some embodiments, filler peptide amphiphiles do not comprise a bioactive segment.

In some embodiments, the nanofiber comprises 5-50% GDNF mimetic peptide amphiphiles and 50-95% filler peptide amphiphiles. In some embodiments, the nanofiber comprises about 12% to about 28% GDNF mimetic peptide amphiphiles and about 72% to about 88% filler peptide amphiphiles. In some embodiments, the nanofiber comprises about 15% GDNF mimetic peptide amphiphiles and about 85% filler peptide amphiphiles.

In some aspects, provided herein is a composition comprising a GDNF mimetic peptide amphiphile or nanofiber provided herein. In some embodiments, the composition of is administered for the subject for treatment of a nervous system injury or a disease. In some embodiments, the disease is Parkinson's disease. In some embodiments, the composition is administered to the subject concurrently with cell replacement therapy.

In some aspects, provided herein is a scaffold comprising a nanofiber provided herein. In some embodiments, provided herein is a cell or an organoid cultured on the scaffold. In some embodiments, the cell is a neuron. In some embodiments, the organoid is a brain organoid.

In some aspects, provided herein is a system comprising a scaffold provided herein and a cell or an organoid cultured on the scaffold. In some embodiments, the cell is a neuron. In some embodiments, the organoid is a brain organoid.

The systems and scaffolds herein find use in treating nervous system injury or disease. For example, in some embodiments provided herein is a method of treating Parkinson's disease in a subject, comprising administering to the subject a system or scaffold provided herein.

### BRIEF DESCRIPTION OF THE DRAWINGS

FIGS. 1A-1G show structural characterization of GDNF mimetic supramolecular nanostructures. Chemical structures of (FIG. 1A) E₂ backbone PA (C16-VVAAEE (SEQ ID NO: 10), Scr PA (C16-VVAAEELRNKSRILS (SEQ ID NO: 18)) and GDNF PA (C16-VVAAEEILKNLSRSR (SEQ ID NO: 17)). (FIG. 1B) Molecular graphics representation of the bioactive supramolecular nanostructure with colors corresponding to the various peptide domains shown in (A) (hydrophobic tail (black), β-sheet domain (red), charged domain (blue) and bioactive domain (green). (FIG. 1C) Cryo-TEM images of E₂ PA, 15 vol % GDNF PA and 15 vol % Scr PA. (FIG. 1D) Small-angle X-ray scattering patterns of nanostructures containing different co-assembly v/v ratios of Scr PA and GDNF PA in the range 15-100 vol %. (FIG. 1E) Wide angle X-ray scattering scans shown as intensity versus the scattering vector q (log-log plot) of Scr PA and GDNF PA at 15-100 vol %. (FIG. 1F) Storage modulus of E₂ PA, 15 vol % Scr PA and 15 vol % GDNF PA. *p<0.05. One-way ANOVA followed by Tukey's multiple comparison test. Data are presented as mean±standard error of the mean (SE). (FIG. 1G) SEM images of neurons seeded on E₂ PA, 15 vol % Scr PA and 15 vol % GDNF PA coatings. Images shows the interaction of neurons and neurites with the nanofibers.

FIGS. 2A-2J show effects of GDNF PA on hiPSC-derived dopaminergic neurons. (FIG. 2A) Schematic diagram illustrating the addition of PA nanofibers. (FIG. 2B) Representative images of neurons treated with poly-L-ornithine+laminin, E₂ PA+100 ng/ml GDNF protein, E₂ PA+10 ng/ml GDNF protein, E₂ PA+100 ng/ml peptide, E₂ PA, 15 vol % GDNF PA, 15 vol % Src PA stained for calcein am (green) and propidium iodide (red) at 2 DIV. (FIG. 2C) Percentage of viable neurons at 2 DIV. *p<0.05, ****p<0. One-way ANOVA followed by Tukey's multiple comparison test; data are presented as mean±SE. (FIG. 2D) Representative images of neurons immunostained for TH⁺ (green) and Tuj1⁺ (orange). (FIG. 2E) TH+ neurite length of neurons at 2 DIV. ****p<0.0001. One-way ANOVA followed by Tukey's multiple comparison test; data are presented as mean±SE. (experiments by triplicate, n=200 cells) (FIG. 2F) Western blot of p-RET, total RET, p-ERK, total ERK and Fyn 10 min post-treatment in neurons treated with starvation (Strv) media, 100 ng/ml GDNF protein, 100 ng/ml peptide, E₂ PA, 15 vol % GDNF PA and 15 vol % Scr PA. Densitometry analysis of the ratio of p-RET/total RET (FIG. 2G), and pERK1/2/total ERK1/2 (FIG. 2H). **p<0.01,*** p<0.001, ****p<0.0001. One-way ANOVA followed by Tukey post hoc test; data are presented as mean±SE (experiments were done in triplicate). Confocal (FIG. 2I) and 3D-rendered image (FIG. 2J) showing the internalization of labeled 15 vol % GDNF PA-TAMRA (red) into neurons stained with Cholera toxin B (cyan) after 10 min, arrowheads show GDNF PA inside the neuron.

FIGS. 3A-3H show effects of RET and NCAM on hiPSC-derived dopaminergic neurons treated with GDNF and GDNF PA. (FIG. 3A) Representative images of neurons treated with GDNF protein (100 ng/ml) and 15 vol % GDNF PA, PP2(P) (Fyn inhibitor) (1 μM)+GDNF or GDNF PA, and Vandetanib(V) (RET inhibitor) (1 μM)+GDNF or GDNF PA stained for calcein am (green) and propidium iodide (red) at 3 DIV. (FIG. 3B) Percentage of viable neurons treated with previous conditions. ****p<0.0001. One-way ANOVA followed by Tukey's multiple comparison test; data are presented as mean±SE. (FIG. 3C) Confocal images of dopaminergic neurons treated with the conditions mentioned previously stained with TH antibody. (FIG. 3D) Neurite length of dopaminergic neurons. ****p<0.0001. One-way ANOVA followed by Tukey's multiple comparison test; data are presented as mean±SE (experiments by triplicate, n=200 cells). (FIG. 3E) Western blot of p-RET, total RET, p-ERK, total ERK and Fyn 10 min post-treatment in neurons treated with the conditions mentioned in (a). Densitometry analysis of the ratio of p-RET/total RET (FIG. 3F), pERK1/2/total ERK1/2 (FIG. 3G) and pFyn/Fyn (FIG. 3H). *p<0.05, **p<0.01, *** p<0.0001, ****p<0.0001. One-way ANOVA followed by Tukey's multiple comparison test; data are presented as mean±SE (experiments by triplicate).

FIGS. 4A-4I show bulk-RNA transcriptomics and electrophysiological characterization of hiPSC-derived dopaminergic neurons treated with GDNF PA. (FIG. 4A) Volcano plot depicting up- and downregulated genes of human dopaminergic neurons cultured on 15 vol % GDNF PA vs. 15 vol % Scr PA for 4 DIV (Fold change cutoff ±1, P-value <0.05). Red dots indicate upregulated genes whereas blue dots indicate downregulated genes. Y-axis denotes −log₁₀ P values while X-axis shows log₂ fold change values. (FIG. 4B) Bar graph of the most significant up (red) and down (blue) enriched GO terms of genes expressed in human dopaminergic neurons cultured on 15 vol % GDNF PA vs. 15 vol % Scr PA for 4 DIV using the SynGO library. (FIG. 4C) Most significant molecular function (MF) GO terms of the upregulated protein-coding genes of human dopaminergic neurons cultured on 15 vol % GDNF PA vs. 15 vol % Scr PA for 4 DIV (P-value <0.05) (n=4 libraries). (FIG. 4D, FIG. 4E, and FIG. 4F) Images and representative recordings of cell-attached patched hiPSC-derived dopaminergic neurons seeded on 15 vol % Scr PA (d) and 15 vol % GDNF PA (e) and PDL with GDNF protein (100 ng/ml) (f). (FIG. 4G) Bar graph showing the percentage of cells that revealed spontaneous activity. Box plots depicting firing rate (FIG. 4H) and coefficient of variation (CV) (FIG. 4I) of cells seeded on 15 vol % Scr PA (i) and 15 vol % GDNF PA (one-way ANOVA followed by Tukey's multiple comparison test; data are presented as mean±SE) (experiments were done in triplicate, GDNF PA n=30, Scr PA n=24 and GDNF protein n=30 cells).

FIGS. 5A-5O show dopaminergic neuron morphology is influenced by GDNF PA scaffolds. (FIG. 5A) Schematic diagram illustrating the procedure for cell encapsulation. (FIG. 5B) Three-dimensional reconstruction of a 500 μm-thick sample using multi-photon microscopy of cells encapsulated in PA scaffolds at 15 DIV; the color scale bar indicates the depth at which the cells are located within the PA scaffold. (FIG. 5C, FIG. 5D) 350 μm thick three-dimensional reconstruction of neurons stained with TH and MAP2 antibodies at 15 DIV encapsulated in a 15 vol % Src PA scaffold (c) and a 15 vol % GDNF PA scaffold (d). (FIG. 5E, FIG. 5F) Quantification of MAP2⁺ (e) and TH+/MAP2⁺ (f) neurons within PA scaffolds. (FIG. 5G, FIG. 5H) Volume of the fluorescence signal of MAP2⁺ neurons (g) and TH⁺/MAP2⁺ neurons (h) within PA scaffolds; *p<0.05; T-test; data are presented as mean±SE. (FIG. 5I, FIG. 5J) Digital reconstruction of single TH⁺ neuron encapsulated in 15 vol % 15 Scr PA (i) and 15 vol % GDNF PA scaffolds (j). Sholl analysis of TH⁺ neurons encapsulated in PA scaffolds (k); *p<0.05, **p<0.01, ***p<0.001, ****p<0.0001; one-way ANOVA followed by Tukey post hoc test; data are presented as mean±SE. Quantification of TH⁺ neurite length (FIG. 5L), volume of TH⁺ fluorescence signal (FIG. 5M), number of neurite branching points (FIG. 5N) and terminal points per TH⁺ neuron (FIG. 5O); **p<0.01, ****p<0.0001; T-test; data are presented as mean±SE (experiments were done triplicate, n=60 cells).

FIGS. 6A-6J show the effect of GDNF PA on the expression of synaptic markers of encapsulated hiPSC-derived dopaminergic neurons. (FIG. 6A, FIG. 6B) Representative fluorescence micrographs of TH+(green) and (Syn⁺) (red) neurites in hiPSC-derived dopaminergic neurons encapsulated in 15 vol % Src PA (a) and 15 vol % GDNF PA scaffolds (b) (Syn⁺ puncta indicated by arrow heads). (FIG. 6C, FIG. 6D) Representative fluorescence images of TH⁺ (green) and (PSD95) (red) neurites in hiPSC-derived dopaminergic neurons encapsulated in 15 vol % Src PA (c) and 15 vol % GDNF PA (d) scaffolds (PSD95⁺ puncta indicated by arrow heads). (FIG. 6E) Quantification of Syn⁺ puncta in TH⁺ neurites of cells encapsulated in 15 vol % Scr PA scaffolds shown in (a) and in 15 vol % GDNF PA scaffolds shown in (b) (*p<0.05; T-test) data are presented as mean±SE. (FIG. 6F) Quantification of PSD95 puncta in TH⁺ neurites of hiPSC-derived dopaminergic neurons encapsulated in 15 vol % Scr PA scaffolds shown in (c) and in 15 vol % GDNF PA scaffolds shown in (d) (****p<0.0001; T-test; data are presented as mean±SE). (FIG. 6G, FIG. 6H) Representative confocal images of dopaminergic (white arrowheads) and non-dopaminergic (yellow arrowheads) neurons embedded in 15 vol % GDNF PA. Number of Syn (FIG. 6I) and PSD95 (FIG. 6J) puncta in TH+ and TH− neurites. (****p<0.0001; T-test; data are presented as mean±SE) (experiments were done triplicate, n=60 cells).

FIGS. 7A-7N show the effect of GDNF PA on the dopaminergic axonal length of hMLOs. (FIG. 7A, FIG. 7B) Cryosection of hMLOs at (a) 45 days stained for Nestin (red) and TH (green) and (b) at 60 days stained for Tuj1 (red) and TH (green). Bottom left: a zoom-in view of the white box. Bottom right: a zoom-in view of the bottom left image. (FIG. 7C) Percentage of cells showing co-localization of TH+/Hoechst+ and Nestin+/TH+ cells at 45 days, and (FIG. 7D) TH+/Hoechst+ and Nestin+/TH+ at 60 days (data are presented as mean±SE). (FIG. 7E) Immunostaining of hMLOs at 80 days showing the maturation markers MAP2, GIRK2 and DAT, arrowheads indicate the double-positive cells. (FIG. 7F, FIG. 7G) Percentage of cells showing co-localization of TH+/Hoechst+ and MAP2+/TH+ (f), GIRK2+/TH+ and DAT/TH+(g) (data are presented as mean±SE). (FIG. 7H) Schematic diagram illustrating the embedding process of the organoid within the PA scaffold. (FIG. 7I) Epifluorescence image to show the top-view of an organoid stained for Tuj1 encapsulated within the PA scaffold. (FIG. 7J) Organoid encapsulated stained for TH and Tuj1. (FIG. 7K-FIG. 7L) Representative images of organoids stained for TH embedded in 15 vol % Scr PA (k) and 15 vol % GDNF PA (1). Zoom-in view of the white box shows confocal images and digital reconstruction of dopaminergic axons. (FIG. 7M) TH+ neurite length and (FIG. 7N) number of TH+ projections of hMLOs embedded within PA scaffolds at 7 DIV (****p<0.0001; T-test; data are presented as mean±SE) (experiments were done triplicate, n=9 organoids).

FIGS. 8A-8L show the neuroprotective effect of GDNF PA against the neurotoxin 6-OHDA. (FIG. 8A) Schematic diagram illustrating the addition of 6-OHDA to the cells seeded on PA coatings. (FIG. 8B) Representative images of hiPSC-derived dopaminergic neurons at 15 DIV treated with 6-OHDA (100 μM) for 1 h seeded on PDL with starvation (Strv) media and with basal media, E₂ PA+100 ng/ml GDNF protein, E₂ PA+100 ng/ml Peptide, E₂ PA, 15 vol % GDNF PA and 15 vol % Scr PA immunostained for TH. (FIG. 8C) Mean intensity of TH fluorescence and (FIG. 8D) TH⁺ neurite length of cells seeded on the different conditions (***p<0.001, ****p<0.0001; one-way ANOVA followed by Tukey's multiple comparison test; data are presented as mean±SE). (FIG. 8E) Western blot and (FIG. 8F, FIG. 8G) densitometry analysis at 15 DIV of (f) Tuj1 and (g) TH in neurons treated with 6-OHDA for 1 h exposed to conditions in (b) (**p<0.01, ***p<0.001, ****p<0.0001); one-way ANOVA followed by Tukey post hoc test; data are presented as mean±SE. (FIG. 8H) Schematic diagram illustrating the addition of 6-OHDA to the cells encapsulated within PA scaffolds. (FIG. 8I, FIG. 8J, FIG. 8K) Three-dimensional reconstruction of neurons (stained with TH) at 15 DIV after treatment with 6-OHDA encapsulated within E₂ PA (i) 15 vol % Scr PA and (j) 15 vol % GDNF PA (k). (FIG. 9L) Quantification of TH⁺ fluorescence volume of neurons encapsulated (*p<0.05, **p<0.01); one-way ANOVA followed by Tukey's multiple comparison test; data are presented as mean±SE) (experiments were done triplicate, n=60 cells).

FIG. 9A-9C show AFM, CD and FT-IR of E2, GDNF and Scr PA nanostructures. (FIG. 9A) Liquid AFM images of E2 PA, 15 vol % GDNF PA, 15 vol % Scr PA from left to the right (scale bar: 220 nm), (FIG. 9B) CD spectra of the E2 PA (black line), E2+ 15 vol % GDNF PA (red line), and E2+ 15 vol % Scr PA (blue line) and (FIG. 9C) FT-IR of the E2 PA (black line), 15 vol % GDNF PA (red line), and 15 vol % Scr PA (blue line).

FIGS. 10A-10C show Cryo-TEM and SEM of PA nanostructures at different co-assembly ratios. (FIG. 10A) Cryo-TEM images of GDNF PA and Scr PA at 100, 80, 50 and 33 volume percent co-assembly ratios. (FIG. 10B) SEM images of E2PA, 15 vol % GDNF PA and 15 vol % Scr PA coatings. (FIG. 10C) Low-magnification SEM images of cells seeded on E2 PA, 15 vol % GDNF PA and 15 vol % Scr PA coatings.

FIGS. 11A-11E show rheological characterization of PAs. (FIG. 11A) Frequency sweep showing the storage moduli, G′, and loss moduli, G″, with angular frequencies ranging from 0-100 rad/s for the different PAs. (FIG. 11B) tan δ (G″/G′) E₂ PA (blue), 15 vol % GDNF PA (red) and 15 vol % Src PA (orange), data are presented as mean±standard deviation of the mean. (FIG. 11C, FIG. 11D, FIG. 11E) Strain sweep showing G′ and G″ at shear strain ranging from 0-100% for (c) E₂ PA, (d) 15 vol % Scr PA and (e) 15 vol % GDNF PA.

FIGS. 12A-12E show cell viability of hiPSC-derived neurons seeded on PA coatings at 2 DIV and 7 DIV. (a) Schematic diagram illustrating the preparation procedure for PA coatings for cell culture experiments. (b-c) Representative fluorescence confocal images of hiPSC-derived neurons seeded on E₂PA, E₂PA+100 ng/ml Peptide, E₂PA+(3.3 nM) Peptide, E₂ PA+10 ng/ml GDNF protein, E₂ PA+100 ng/ml (3.3 nM) GDNF protein, 10 vol % Scr PA, 10 vol % GDNF PA, 15 vol % Scr PA, 15 vol % GDNF, 33 vol % Scr PA, 33 vol % GDNF PA and E₂PA+GDNF protein 10 ng/ml at (b) 2 DIV and (c) 7 DIV, calcein (green) and propidium iodide (red). (d-e) Percentage of cell viability of hiPSC-derived neurons based on calcein (green) and propidium iodide (red) assay at (d) 2 DIV and (e) 7 DIV; (*p<0.05, **p<0.01***p<0.001, ****p<0.0001; one-way ANOVA followed by Tukey's multiple comparison test; data are presented as mean±standard error of the mean) (experiments were done in triplicate, n=200 cells).

FIG. 13A-13F show dopaminergic neurite length of hiPSC-derived neurons seeded on PA coatings at 2DV and 7 DIV. (FIG. 13A, FIG. 13B) Representative fluorescence confocal images of hiPSC-derived neurons seeded on E₂PA, E₂PA+100 ng/ml Peptide, E₂PA+(3.3 nM) Peptide, E₂ PA+10 ng/ml GDNF protein, E₂ PA+100 ng/ml (3.3 nM) GDNF protein, 10 vol % Scr PA, 10 vol % GDNF PA, 15 vol % Scr PA, 15 vol % GDNF, 33 vol % Scr PA, 33 vol % GDNF PA and Poly-D-lysine at (a) 2 DIV and (b) 7 DIV, stained for TH (green) and MAP2 (red). (FIG. 13C, FIG. 13D) Percentage of TH+ neurite length of hiPSC-derived neurons (c) 2 DIV and (d) 7 DIV; (*p<0.05, **p<0.01, ****p<0.0001; one-way ANOVA followed by Tukey's multiple comparison test; data are presented as mean±standard error of the mean). (FIG. 13E) Representative images of neurons seeded on PA coatings and PDL showing the number of intersections obtained from Sholl analysis (color scale bar indicating the number of intersections). (FIG. 13F) Sholl profiles of neurons cultured in various conditions (*p<0.05, **p<0.01, ***p<0.001); one-way ANOVA followed by Tukey's multiple comparison test; data are presented as mean±standard error of the mean. (experiments were done in triplicate, n=200 cells)

FIG. 14 shows GDNF PA internalization at 2 DIV and 4 DIV. Representative confocal and IMARIS-digital images of neurons (cyan) exposed to labeled GDNF PA (red) at 2 DIV (10 min and 1 hr) and 7 DIV (10 min and 1 hr), arrowheads indicate PA inside the neurons.

FIG. 15A-15C show genes and GO terms upregulated on human dopaminergic neurons cultured on GDNF PA coatings. (FIG. 15A) Bar graph of the 20 most upregulated and downregulated protein-coding genes of human dopaminergic neurons cultured on 15 vol % GDNF PA vs. 15 vol % Scr PA for 4 DIV (P-value <0.05). X-axis shows log₂ fold change. FIG. 15B Most significant molecular function GO terms of human dopaminergic neurons cultured on 15 vol % GDNF PA vs. 15 vol % Scr PA for 4 DIV (P-value <0.05). (FIG. 15C) Venn diagram showing the overlapping of genes with a fold change higher than 1 between human dopaminergic neurons cultured on GDNF PA coatings and human ventral midbrain progenitors grafted in a GDNF-enriched environment, as published by Gantner et. al (Gantner, Carlos W. et al. Cell Stem Cell, Volume 26, Issue 4, 511-526.e5).

FIGS. 16A-16E show encapsulation of hiPSC-derived dopaminergic neurons in PA scaffolds. (FIGS. 16A-16D) Representative three-dimensional fluorescence reconstructions of hiPSC-derived neurons encapsulated in 15 vol % Scr PA (FIGS. 16A-16B) and 15 vol % GDNF PA (FIGS. 16C-16D) scaffolds stained for TH and MAP2. (FIG. 16E) Bar graph of fluorescence intensity of TH marker distributed within 350 μm thick PA scaffolds, data are presented as mean±standard error of the mean.

FIGS. 17A-17F show fluorescence intensity of synaptic markers of hiPSC-derived dopaminergic neurons encapsulated. (FIG. 17A, FIG. 17B) Representative fluorescence images and fluorescence plot profile of hiPSC-derived dopaminergic neurons encapsulated in 15 vol % Src PA (a) and 15 vol % GDNF PA (b) scaffolds stained for synaptophysin (Syn⁺). (FIG. 17C, FIG. 17D) Representative fluorescence images and plot profile of fluorescence intensity of hiPSC-derived dopaminergic neurons encapsulated in 15 vol % Src PA (c) and 15 vol % GDNF PA (d) scaffolds stained for PSD95. Quantification of fluorescence intensity signal of Syn⁺ (FIG. 17E) and PSD95⁺ (FIG. 17F) puncta of hiPSC-derived dopaminergic neurons encapsulated in 15 vol % GDNF PA and 15 vol % Scr PA (****p<0.0001; T-test; data are presented as mean±standard error of the mean.) (experiments were done in triplicate, n=60 cells).

FIGS. 18A-18B show human midbrain-like organoids embedded in PA scaffolds. (FIG. 18A) Experiment set-up of organoids embedded on PA scaffolds in a 4-well chamber. (FIG. 18B) Three-dimensional reconstruction of a 500 μm-thick sample of midbrain-like organoids encapsulated in Scr PA and GDNF PA scaffolds; the color scale bar indicates the depth; arrowheads indicate axons growing through the PA.

FIGS. 19A-19H show expression of Cleaved-Caspase 3 on hiPSC-derived dopaminergic neurons encapsulated post 6-OHDA. (FIG. 19A, FIG. 19B, FIG. 19C) Representative fluorescence confocal images of hiPSC-derived dopaminergic neurons seeded on E₂PA (a), 15 vol % Scr PA (b) and 15 vol % GDNF PA (c), and then treated with 6-OHDA (100 μM) for 1 hr, stained for TH and Cleaved-Caspase 3. (FIG. 19D, FIG. 19E, FIG. 19F) Representative 3D fluorescence reconstructions of hiPSC-derived neurons encapsulated in E₂PA (d), 15 vol % Scr PA (e) and 15 vol % GDNF PA (f) scaffolds and then treated with 6-OHDA (100 μM) for 1 hr. Bar graphs showing the percentage of co-localization between TH⁺ and Cleaved-Caspase 3⁺ neurons cultured on the coatings (FIG. 19G) and scaffolds (FIG. 19H) described in (a-c) and (d-f) respectively (*p<0.05, ****p<0.0001; one-way ANOVA followed by Tukey's multiple comparison test; data are presented as mean±standard error of the mean.) (experiments were done in triplicate, n=60 cells).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

The terms “GDNF mimetic”, “GDNF mimetic peptide”, “GDNF mimetic sequence”, and “GDNF mimetic peptide sequence” are used interchangeably herein and refer to a peptide sequence that mimics one or more biological activities of glial cell derived neurotrophic factor (GDNF).

As used herein, the term “nanofiber” refers to an elongated or threadlike filament (e.g., having a significantly greater length dimension that width or diameter) with a diameter typically less than 100 nanometers.

As used herein, the term “scaffold” refers to a material capable of supporting growth and differentiation of a cell.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a hydrophobic segment, a structural peptide segment, and a charged peptide segment. In some embodiments, a peptide amphiphile additionally comprises a bioactive segment, such as a GDNF mimetic peptide sequence. The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment; (3) a charged peptide segment, and (4) a bioactive segment (e.g., a GDNF mimetic sequence).

The term “peptide amphiphile” is inclusive of both “bioactive peptide amphiphiles” and “filler peptide amphiphiles” or “diluent peptide amphiphiles”. A “bioactive peptide amphiphile” refers to a peptide amphiphile comprising a bioactive segment, such as a GDNF mimetic peptide sequence. A “GDNF mimetic peptide amphiphile” is an example of a “bioactive peptide amphiphile”. In contrast, a filler or diluent peptide amphiphile does not comprise a bioactive segment (e.g. does not comprise a GDNF mimetic peptide sequence). As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)—where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used interchangeably herein, the terms “structural peptide” or “structural peptide segment” refer to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural peptide segments of adjacent structural peptide segments. In some embodiments, nanofibers of peptide amphiphiles having structural peptide segments display linear or 2D structure when examined by microscopy and/or α-helix and/or β-sheet character when examined by circular dichroism (CD). In some embodiments, the structural peptide segment has a propensity for forming β-sheet conformations. Such a structural peptide segment is also referred to herein as a “beta (β)-sheet-forming peptide segment”. In some embodiments, the structural peptide segment comprises 2 to 8 non-polar residues. In some embodiments, the structural peptide segment comprises 2 to 8 valine and/or alanine residues. In some embodiments, the structural peptide comprises V₂A₂ (SEQ ID NO: 2).

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), non-natural amino acids, or peptidomimetics). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., a GDNF mimetic peptide sequence) exhibit the functionality of the bioactive peptide.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

As used herein, the terms “co-administration” and “co-administering” refer to the administration of at least two agent(s) or therapies to a subject (e.g., a PA nanofiber and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are bioactive peptide amphiphiles (PAs) comprising a GDNF mimetic peptide sequence, nanofibers comprising the bioactive PAs (e.g. nanofibers displaying the GDNF mimetic peptide), and methods of use thereof.

In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles comprise a hydrophobic segment (i.e. a hydrophobic tail) linked to a peptide. In some embodiments, the peptide comprises a structural peptide segment. In some embodiments, the structural peptide segment is a hydrogen-bond-forming segment, or beta-sheet-forming segment. In some embodiments, the structural peptide segment has the propensity to form random coil structures (e.g. a total propensity for forming n-sheet conformations of 4 or less). In some embodiments, the peptide comprises a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) to bury the lipophilic segment in their core and display the bioactive peptide (e.g. GDNF mimetic sequence) on the surface. In some embodiments, the structural peptide undergoes intermolecular hydrogen bonding to form beta sheets that orient parallel to the long axis of the micelle. In some embodiments, the structural peptide displays weak intermolecular hydrogen bonding, resulting in a less rigid beta-sheet conformation within the nanofibers.

In some embodiments, compositions described herein comprise PA building blocks that in turn comprise a hydrophobic segment (also referred to as a hydrophobic tail) and a peptide segment (e.g. a structural peptide segment and/or a charged peptide segment). In certain embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between.) is covalently coupled to peptide segment (e.g., a peptide comprising a segment having a preference for beta-strand conformations or other supramolecular interactions) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30).

In some embodiments, PAs comprise one or more peptide segments. A peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises E₂-4. For example, in some embodiments an acidic peptide segment comprises EE. In some embodiments, an acidic peptide segment comprises EEE. In other embodiments, an acidic peptide segment comprises EEEE (SEQ ID NO: 4).

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K. For example, in some embodiments the basic peptide segment comprises KK, KKK, or KKKK (SEQ ID NO: 9).

In some embodiments, peptide amphiphiles comprises a structural peptide segment. In some embodiments, the structural peptide segment is a beta-sheet-forming segment. In some embodiments, the structural peptide segment displays weak hydrogen bonding and has the propensity to form random coil structures rather than rigid beta-sheet conformations. In some embodiments, the structural peptide segment is rich in one or more of H, I, L, F, V, G, and A residues. In some embodiments, the structural peptide segment comprises an alanine- and valine-rich peptide segment (e.g., VVAA (SEQ ID NO: 2), VVVAAA (SEQ ID NO: 3), AAVV (SEQ ID NO: 5), AAAVVV (SEQ ID NO: 6), VVAAA (SEQ ID NO: 7), VVVAA (SEQ ID NO: 8), or other combinations of V and A residues, etc.). In some embodiments, the structural peptide segment comprises 4 or more consecutive A and/or V residues, or conservative or semi-conservative substitutions thereto. In some embodiments, the structural peptide segment comprises V₂A₂(SEQ ID NO: 2).

In some embodiments, the structural peptide segment comprises an alanine and glycine-rich peptide segment (e.g. AAGG (SEQ ID NO: 11), AAAGGG (SEQ ID NO: 12), or other combinations of A and G residues, etc.). In some embodiments, the structural peptide segment comprises A₂G₂(SEQ ID NO: 11). In some embodiments, the structural peptide segment comprises GGGG (SEQ ID NO: 13).

In some embodiments, the structural peptide segment comprises 4 or more consecutive non-polar aliphatic residues (e.g., alanine (A), valine (V), leucine (L), isoleucine (I), methionine (M)). In some embodiments, the structural peptide segment comprises 2-16 amino acids in length and comprises 4 or more (e.g., 2, 3, 4, 5, 6, 7, 8, 9, 10, 11, 12, 13, 14, 15, 16, or ranges there between) non-polar aliphatic residues. In some embodiments, the structural peptide segment comprises VEVA₂ (SEQ ID NO: 14).

In some embodiments, the structural peptide segment has a total propensity for forming β-sheet conformations of 4 or less (e.g. less than 4, less than 3.9, less than 3.8, less than 3.7, less than 3.6, less than 3.5, less than 3.4, less than 3.3, less than 3.2, less than 3.1, less than 3.0, less than 2.9. less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, or less than 1.)

In some embodiments, the structural peptide segment has a total propensity for forming β-sheet conformations of 4 or more. For example, in some embodiments the structural peptide segment has a total propensity for forming β-sheet conformations of at least 4, at least 4.1, at least 4.2, at least 4.3, at least 4.4, at least 4.5, at least 4.6, at least 4.7, at least 4.8, at least 4.9, at least 5.0, at least 5.1, at least 5.2, at least 5.3, at least 5.4, at least 5.5, at least 5.6, at least 5.7, at least 5.8, at least 5.9, or at least 6.

The total propensity for forming β-sheet conformations may be calculated as the sum of the propensity for forming β-sheet conformations of each amino acid in the structural peptide segment. The propensity of each amino acid for forming β-sheet conformations and methods for calculating the same are described in, for example, Fujiwara, K., Toda, H. & Ikeguchi, M. Dependence of α-helical and β-sheet amino acid propensities on the overall protein fold type. BMC Struct Biol 12, 18 (2012), the entire contents of which are incorporated herein by reference. Exemplary values are shown in Table 1, below. For the purposes of calculating the total propensity for forming β-sheet conformations of the structural peptide segment, the value shown in the “total residues” column from table 1 for each amino acid is added together. For example, for an A₂G₂(SEQ ID NO: 11) structural peptide segment, the total propensity for forming β-sheet conformations is 0.75+0.75+0.67+0.67=2.84. The structural peptide segment may comprise any suitable number and combination of amino acids to achieve a total propensity for forming β-sheet conformations of 4 or less.

TABLE 1
Amino acid Propensities for β-sheet conformations

| Amino Acid | Exposed Residues | Buried Residues | Total Residues |
|---|---|---|---|
| V | 2.31 | 1.57 | 2.00 |
| I | 2.02 | 1.39 | 1.79 |
| L | 1.18 | 0.93 | 1.15 |
| M | 1.01 | 0.84 | 1.01 |
| P | 0.49 | 0.42 | 0.40 |
| A | 0.48 | 0.72 | 0.75 |
| C | 1.24 | 1.07 | 1.36 |
| F | 1.50 | 1.10 | 1.4 |
| Y | 1.71 | 1.12 | 1.37 |
| W | 1.90 | 0.91 | 1.23 |
| Q | 0.96 | 0.82 | 0.72 |
| S | 0.86 | 0.85 | .081 |
| T | 1.58 | 1.08 | 1.21 |
| N | 0.71 | 0.76 | 0.63 |
| H | 1.15 | 0.98 | 0.99 |
| D | 0.61 | 0.76 | 0.55 |
| K | 1.14 | 0.98 | 0.76 |
| E | 0.89 | 0.86 | 0.65 |
| R | 1.27 | 0.82 | 0.85 |
| G | 0/41 | 0.81 | 0.67 |

In some embodiments, a structural peptide segment having a total propensity for forming β-sheet conformations of 4 or less indicates that the amino acids within the structural peptide segment have weaker interactions with neighboring molecules. For example, the structural peptide segment may display weak hydrogen-bonding abilities. Accordingly, such structural peptide segments and the peptide amphiphiles comprising the same may create more dynamic nanofiber structures. For example, an A₂G₂(SEQ ID NO: 11) structural peptide segment may display random coil structures rather than rigid beta-sheet conformations.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group (e.g. a GDNF mimetic peptide sequence). In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH2(O(CH₂)₂)₂NH, CH2(O(CH₂)₂)₂NHCO(CH₂)₂CCH, etc.). In some embodiments, a spacer or linker further comprises additional bioactive groups, substituents, branches, etc. In some embodiments, the linker segment is a single glycine (G) residue. In some embodiments, the linker comprises GG. In some embodiments, the linker comprises GGG. In some embodiments, the linker is a single lysine residue (K).

Suitable peptide amphiphiles for use in the materials herein, as well as methods of preparation of PAs and related materials, amino acid sequences for use in PAs, and materials that find use with PAs, are described in the following patents and applications: U.S. Pat. Nos. 9,044,514; 9,040,626; 9,011,914; 8,772,228; 8,748,569 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167; and WO2020154631A1; all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural peptide segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural peptide segment (e.g., comprising VVAA (SEQ ID NO: 2)); and (c) a charged segment (e.g., comprising EE, EEE, EEEE (SEQ ID NO: 4), etc.).

In some embodiments, peptide amphiphiles comprise a GDNF mimetic peptide. sequence. A GDNF mimetic peptide is considered a “bioactive moiety”. In particular embodiments, the GDNF mimetic peptide is the most C-terminal or N-terminal segment of the PA. In some embodiments, the GDNF mimetic peptide is attached to the end of the charged segment. In some embodiments, the GDNF mimetic peptide is exposed on the surface of an assembled PA structure (e.g., nanofiber).

In some embodiments, the GDNF mimetic peptide comprises a sequence having at least 75% identity to ILKNLSRSR (SEQ ID NO: 1). In some embodiments, the GDNF mimetic peptide comprises a sequence having at least 88% identity to ILKNLSRSR (SEQ ID NO: 1). In some embodiments, the GDNF mimetic peptide comprises the sequence ILKNLSRSR (SEQ ID NO: 1). In some embodiments, the GDNF mimetic peptide consists of a sequence having at least 75% identity to ILKNLSRSR (SEQ ID NO: 1). In some embodiments, the GDNF mimetic peptide consists of a sequence having at least 88% identity to ILKNLSRSR (SEQ ID NO: 1). In some embodiments, the GDNF mimetic peptide consists of ILKNLSRSR (SEQ ID NO: 1).

In some embodiments, a peptide amphiphile comprises (e.g., from C-terminus to N-terminus or from N-terminus to C-terminus): a GDNF mimetic peptide (e.g. ILKNLSRSR (SEQ ID NO: 1))—charged segment (e.g., comprising EE, EEE, EEEE (SEQ ID NO: 4), etc.)—structural peptide segment (e.g., comprising V₂A₂(SEQ ID NO: 2)—hydrophobic tail (e.g., comprising an alkyl chain of 8-24 carbons).

In some embodiments, a peptide amphiphile comprises (e.g., from C-terminus to N-terminus or from N-terminus to C-terminus): GDNF mimetic peptide (e.g. ILKNLSRSR (SEQ ID NO: 1))—flexible linker (e.g. K)—charged segment (e.g., comprising EE, EEE, EEEE (SEQ ID NO: 4), etc.)—structural peptide segment (e.g., comprising V₂A₂(SEQ ID NO: 2)—hydrophobic tail (e.g., comprising an alkyl chain of 8-24 carbons).

In some embodiments, a PA further comprises an attachment segment or residue (e.g., K) for attachment of the hydrophobic tail to the peptide portion of the PA. In some embodiments, the hydrophobic tail is attached to a lysine side chain.

In some embodiments, provided herein are nanofibers and nanostructures assembled from the peptide amphiphiles described herein. In some embodiments, a nanofiber is prepared by the self-assembly of the PAs described herein. In some embodiments, a nanofiber comprises or consists of GDNF mimetic PAs (e.g. PAS comprising the GDNF mimetic peptide). In some embodiments, the GDNF mimetic peptide is displayed on the surface of the nanofiber. In some embodiments, in addition to GDNF mimetic PAs, filler PAs are included in the nanofibers. In some embodiments, filler PAs are peptide amphiphiles, as described herein (e.g., structural peptide segment, charged segment, hydrophobic segment, etc.), but lacking a bioactive segment (e.g. lacking a GDNF mimetic peptide sequence). In some embodiments, filler peptides are basic or acidic peptides lacking a bioactive segment. In some embodiments, the filler PAs and GDNF mimetic PAs self-assemble into a nanofiber comprising both types of PAs. In some embodiments, nanostructures (e.g., nanofibers) assembled from the peptide amphiphiles described herein are provided.

In some embodiments, filler peptides (e.g., basic peptide, acidic peptides, etc.) impart mechanical characteristics to a material comprising the PA nanofibers described herein. In some embodiments, a nanofiber assembled from 0-75% (mass %) GDNF mimetic PA and 25-100% (mass %) basic filler PA becomes a gel at basic pH conditions (e.g., pH 8.5-11). In some embodiments, a nanofiber assembled from 75-100% (mass %) GDNF mimetic PA and 0-25% (mass %) basic filler PA is a liquid at basic pH conditions (e.g., pH 8.5-11). In some embodiments, a nanofiber assembled from 0-20% (mass %) GDNF mimetic PA and 80-100% (mass %) acidic filler PA becomes a gel at acidic pH conditions (e.g., pH 1-5). In some embodiments, a nanofiber assembled from 20-80% (mass %) GDNF mimetic PA and 20-80% (mass %) acidic filler PA becomes a gel at neutral pH conditions (e.g., pH 5-8.5). In some embodiments, a nanofiber assembled from 80-100% (mass %) GDNF mimetic PA and 0-20% (mass %) acidic filler PA is a liquid at acidic pH conditions (e.g., pH 1-5).

In some embodiments, nanostructures are assembled from (1) GDNF mimetic PAs and (2) filler PAs (e.g., acidic or basic PAs not-labeled or not displaying a GDNF mimetic sequence) In some embodiments, nanostructures (e.g., nanofibers) comprise about 5% to about 50% GDNF mimetic peptide amphiphiles and about 50% to about 95% filler peptide amphiphiles. In some embodiments, nanostructures (e.g., nanofibers) comprise about 5% to about 50% GDNF mimetic peptide amphiphiles, about 7.5% to about 40% GDNF mimetic PAs, about 10% to about 30% GDNF mimetic PAs, about 12% to about 30% GDNF mimetic PAs, about 12% to about 28% GDNF mimetic PAs, about 12% to about 26%, about 12% to about 24%, about 12% to about 22%, about 12% to about 20%, about 12% to about 18%, about 12% to about 16%, or about 14% to about 16% GDNF mimetic PAs. In some embodiments, the nanofibers comprise about 11% to about 29% GDNF mimetic PAs. In some embodiments, nanofibers comprise about 11% to about 29%, about 11% to about 28%, about 12% to about 28%, about 12% to about 27%, about 14% to about 26%, or about 15% to about 25% GDNF mimetic PAs. In some embodiments, nanofibers comprise about 10% to about 20%, about 11% to about 19%, about 12% to about 18%, about 13% to about 17%, about 14% to about 16%, or about 15% GDNF mimetic PAs.

In some embodiments, the nanostructures (e.g. nanofibers) comprise about 50% to about 95% filler PAs, about 60% to about 92.5% filler PAs, about 70% to about 90% filler PAs, about 70% to about 88% filler PAs, about 72% to about 88% filler PAs, about 74% to about 88% filler PAs, about 76% to about 88% filler PAs, about 78% to about 88% filler PAs, about 80% to about 88% filler PAs, about 82% to about 88% filler PAs, about 84% to about 88% filler PAs, or about 84% to about 86% filler PAs. In some embodiments, the nanostructures comprise about 85% filler PAs. In some embodiments, the nanofibers comprise about 71% to about 89% GDNF mimetic PAs. In some embodiments, nanofibers comprise about 71% to about 89%, about 72% to about 89%, about 72% to about 88%, about 73% to about 88%, about 74% to about 86%, or about 75% to about 85% filler PAs. In some embodiments, nanofibers comprise about 80% to about 90%, about 81% to about 89%, about 82% to about 88%, about 83% to about 87%, about 84% to about 86%, or about 85% filler PAs.

In some embodiments, the ratio of GDNF mimetic PA to acidic and/or basic PAs in a nanofiber determines the mechanical characteristics (e.g., liquid or gel) of the nanofiber material and under what conditions the material will adopt various characteristics (e.g., gelling upon exposure to physiologic conditions, liquifying upon exposure to physiologic conditions, etc.).

Peptide amphiphile (PA) nanofiber solutions may comprise any suitable combination of PAs. In some embodiments, at least 0.05 mg/mL (e.g., 0.10 mg/ml, 0.15 mg/ml, 0.20 mg/ml, 0.25 mg/ml, 0.30 mg/ml, 0.35 mg/ml, 0.40 mg/ml, 0.45 mg/ml, 0.50 mg/ml, 0.60 mg/ml, 0.70 mg/ml, 0.80 mg/ml, 0.90 mg/ml, 1.0 mg/ml, or more, or ranges therebetween), of the solution is a filler PA (e.g., without a bioactive segment). In some embodiments, at least 0.25 mg/mL of the solution is a filler PA. In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. In some embodiments, a filler PA is a non-bioactive PA molecule having highly charged lysine residues on the terminal end of the molecule (e.g., surface-displayed end). These positively charged PAs allow for the gelation to take place under basic conditions. The filler PAs provide the ability to incorporate other bioactive PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C₁₆V₂A₂E₂(SEQ ID NO: 10) which is herein incorporated by reference in its entirety.

In some embodiments, the PA nanofiber described herein exhibit a small cross-sectional diameter (e.g., <25 nm, <20 nm, <15 nm, about 10 nm, etc.). In some embodiments, the small cross-section of the nanofibers (~10 nm diameter) allows the fibers to permeate the brain parenchyma.

In some embodiments, the PAs, systems, and nanofibers described herein may be incorporated into a composition (e.g. pharmaceutical compositions) for use in methods of treating a disease, disorder, condition, or injury in a subject. In some embodiments, the composition is used in methods of treating a nervous system injury or disorder in a subject. The GDNF mimetic peptide amphiphiles and nanofibers comprising the same are shown herein to promote the growth, maturation, and viability of neurons. As such, in some embodiments compositions comprising a GNDF mimetic PA or nanofiber comprising the same find use in methods of treating an injury or disease in a subject that has resulted in damage to or loss of neurons in the central nervous system, such as stroke, neurodegenerative disease, brain injury, spinal cord injury, Parkinson's disease, and the like. For example, in some embodiments compositions find use in methods of treating Parkinson's Disease, characterized by a loss of dopaminergic neurons in the substantia nigra. In some embodiments, the compositions provided herein are used concurrently with cell replacement therapy to treat a disease, disorder, condition, or injury in the subject (e.g. that has resulted in loss of or damage to neurons in the central nervous system). For example, in some embodiments the subject is a candidate for cell replacement therapy for treatment of Parkinson's disease, and the compositions herein are provided concurrently with the cell replacement therapy to improve outcomes from cell replacement therapy, which is typically hindered by poor survival rates for transplanted cells. For example, in some embodiments provided herein is a method comprising providing a composition provided herein to a subject, wherein the subject has received, will receive, or is receiving cell replacement therapy for treatment of a nervous system injury or disorder. The composition improves outcomes following cell replacement therapy, such as improves survival of transplanted neurons in the subject.

Providing the compositions “concurrently” with cell replacement therapy does not necessarily indicate that the composition is provided simultaneously with cell replacement therapy. “Concurrently” indicates that the composition is provided to the subject within a suitable window of cell replacement therapy to exert a beneficial effect on the cell replacement therapy. For example, “concurrently” can indicate that the composition is provided to the subject within 1 week of cell replacement therapy (e.g. from about 7 days before to about 7 days after cell replacement therapy, such as 7 days before, 6 days before, 5 days before, 4 days before, 3 days before, 2 days before, 1 day before, on the day of, 1 day after, 2 days after, 3 days after, 4 days after, 5 days after, 6 days after, or 7 days after cell replacement therapy). In some embodiments, the composition comprises the GDNF mimetic PA (or a nanofiber comprising the same) and additionally comprises the cells for cell replacement therapy. In such embodiments, a single composition comprising cells for the cell replacement therapy and the GDNF mimetic PA is provided to the subject in need of cell replacement therapy. Such a composition improves outcomes following cell replacement therapy, such as improves the survival of transplanted cells in the subject. In other embodiments, a composition is provided to the subject and cells for cell replacement therapy are provided separately to the subject.

The composition may be administered in any suitable amount, depending on factors including the age of the subject, weight of the subject, severity of the injury, and the like. The composition may be administered in combination with other suitable treatments for injury or preventative measures to prevent the severity of the injury from worsening.

In some embodiments, the PA and nanofiber compositions herein are formulated for delivery to a subject. In some embodiments, the compositions are formulated for parenteral administration (e.g. by injection). Suitable routes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the PA compositions are administered parenterally. In some embodiments, parenteral administration is by intrathecal administration, intracerebroventricular administration, or intraparenchymal administration. The PA compositions herein can be administered as the sole active agent or in combination with other pharmaceutical agents such as other agents used in the treatment of nervous system injury or disease in a subject.

In some embodiments, the PAs and nanofibers described herein find use in cell/organoid culture methods. For example, further disclosed herein are scaffolds (e.g. hydrogels) comprising the peptide amphiphiles described herein. The scaffolds may comprise a nanofiber of self-assembled peptide amphiphiles, at least a portion of the peptide amphiphiles comprising: a hydrophobic tail, a structural peptide segment, a charged peptide segment, and a GDNF mimetic sequence. The scaffold may further comprise one or more filler peptide amphiphiles. The scaffolds described herein are capable of supporting growth and differentiation of a cell. Accordingly, the scaffolds may be in methods for culturing cells or organoids. The methods for culturing cells or organoids comprise contacting the cells or organoids with a scaffold as described here. In some embodiments, the scaffold may be used as a coating for any desired cell culture tool (tissue culture plate, petri dish, glass slide, etc.). In some embodiments, the scaffold is placed on top of cells after culturing the cells on a desired cell culture tool.

Cells or organoids cultured on the scaffolds disclosed herein may demonstrate improved characteristics compared to cells or organoids cultured in the absence of the disclosed scaffolds. For example, cells or organoids may demonstrate improved differentiation, improved maturation and/or improved long term viability compared to cells or organoids cultured in the absence of the disclosed scaffolds. In some embodiments, the scaffolds may be used in methods of culturing neuronal cells (e.g. neurons). In some embodiments, neurons cultured on the scaffolds provided herein display enhanced synaptogenesis and functional maturation (e.g. neurite outgrowth, axonal projections, improved electrical activity (e.g. cell signaling) etc.) compared to neurons cultured on a scaffold lacking the GDNF mimetic PA. In some embodiments, the scaffolds may be used in methods of culturing brain organoids, neural organoids, neurospheroids, and the like.

In some aspects, provided herein is a system comprising a scaffold herein and a cell or an organoid. In some embodiments, the system comprises a scaffold and a neuron cultured on the scaffold, and the system is provided to a subject to treat a nervous system injury or disease in the subject. For example, in some aspects provided herein is a system for cell replacement therapy, the system comprising neurons (e.g. dopaminergic neurons) and a scaffold comprising a GDNF mimetic peptide amphiphile as described herein. In some embodiments, the system is provided to the subject as a cell replacement therapy for a subject in need thereof. In some embodiments, the subject has a disease or an injury that causes loss of neurons in the central nervous system. In some embodiments, the system is provided to a subject having Parkinson's disease.

### EXPERIMENTAL

### Example 1

The cardinal motor symptoms of Parkinson's disease (PD) are caused by the degeneration of dopaminergic neurons, making them in principle amenable to treatment by cell replacement therapy. Although cell replacement therapy has shown promise in animal models, it has not been as successful in clinical trials. One likely reason for this lack of success is the poor survival of grafted neurons. In animal models, glial cell derived neurotrophic factor (GDNF) increases the survival of transplanted dopaminergic neurons, but the ability of GDNF to promote survival is limited by its degradation and undesirable side effects. To overcome this limitation, supramolecular nanofibers that display a GDNF-mimetic peptide on their surfaces were designed and tested herein. This nanostructure mimicked the biological effects of GDNF on human dopaminergic neurons, improving cell survival and promoting genetic, morphological, synaptic and electrophysiological maturation. The GDNF nanostructures also enhanced the morphological maturation of midbrain-like organoids. Thus, the GDNF supramolecular mimic provides a self-assembling matrix that can be delivered with transplanted dopaminergic neurons to enhance their survival and maturation in vivo.

### Results

### Design and Characterization of GDNF Mimetic Peptide Amphiphile Nanostructures

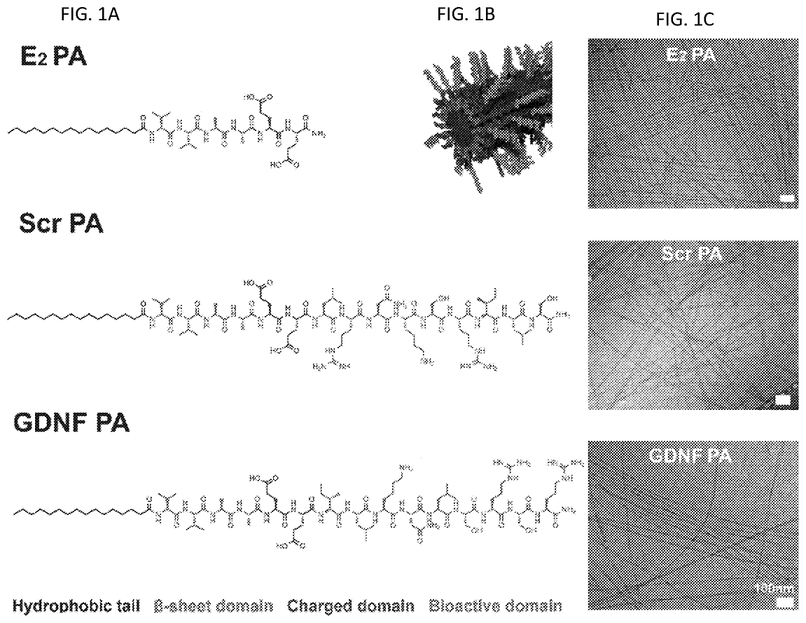

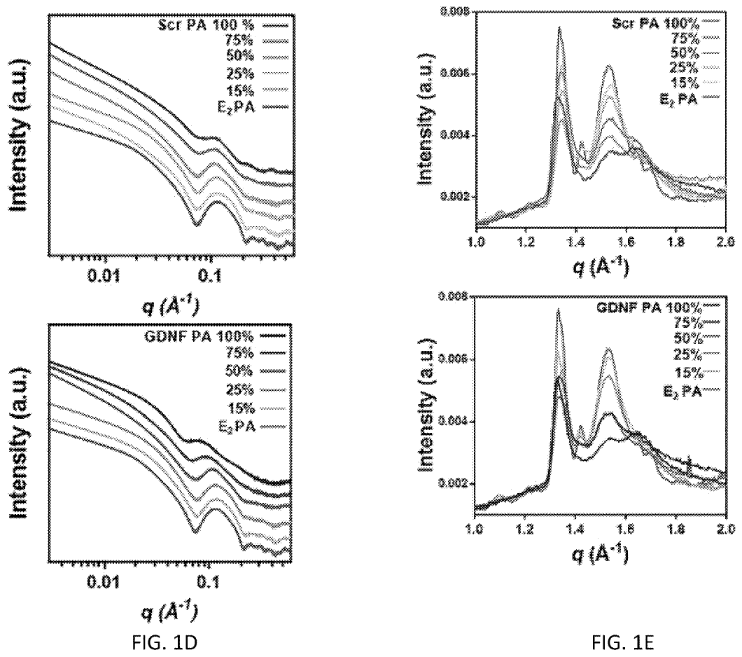

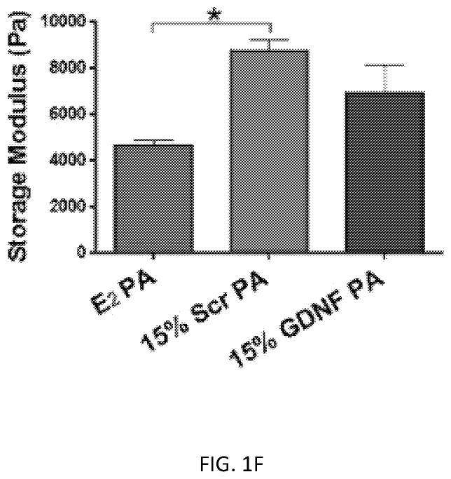

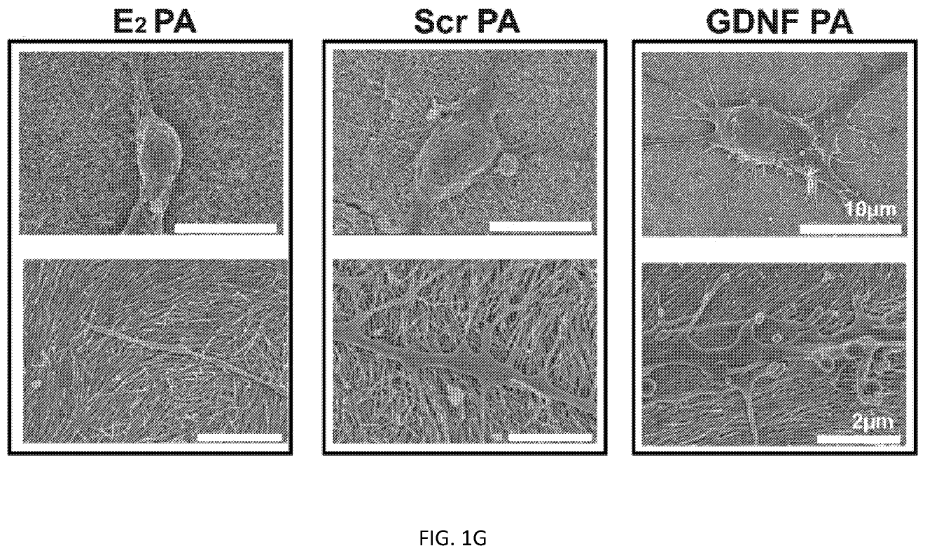

A GDNF-mimetic PA was designed by incorporating the sequence ILKNLSRSR (SEQ ID NO: 1) at the C terminus of the peptide sequence so that it could be displayed on the surfaces of supramolecular nanostructures upon self-assembly of the monomers. To test the specificity of the bioactive peptide sequence, a PA with a scrambled version of the bioactive sequence (LRNKSRILS (SEQ ID NO: 15) at the C terminus of the PA chemical structure was also designed, referred to as the scrambled PA (Scr PA). The PA containing only a non-bioactive peptide sequence consists of a 16-carbon fatty acid tail (palmitic acid), followed by two valine residues, two alanine residues (referred to as the β-sheet domain) and two glutamic acid residues (charged domain) (C₁₆V₂A₂E₂(SEQ ID NO: 10); referred to as E2PA) (FIG. 1a). The bioactive PA nanostructure is shown schematically (FIG. 1b). PAs and peptides were characterized by mass spectrometry and NMR to confirm structure. To determine the co-assembly ratio of E2 PA and the GDNF PA that provides an optimal biological effect, the two PAs were mixed at different volume percentages (vol %) to create supramolecular copolymers (Table 2). All samples were prepared at 1 weight percent (wt %).

TABLE 2
List of PA co-assemblies used

| | | GDNF PA | Total PA | Mol-% |
|---|---|---|---|---|
| % of | % of E2- | concentration | concentration | bioactive |
| GDNF-PA | PA | (mM) | (mM) | epitope |
| 0% | 100% | 0 | 11.71 | 0 |
| 10% | 90% | 0.52 | 11.06 | 4.7 |
| 15% | 85% | 0.78 | 10.73 | 7.3 |
| 25% | 5% | 1.3 | 10.08 | 12.9 |
| 33% | 66% | 1.72 | 9.56 | 17.9 |
| 50% | 50% | 2.6 | 8.45 | 30.8 |
| 75% | 25% | 3.9 | 6.83 | 57.1 |
| 100% | 0% | 5.9 | 5.2 | 100 |

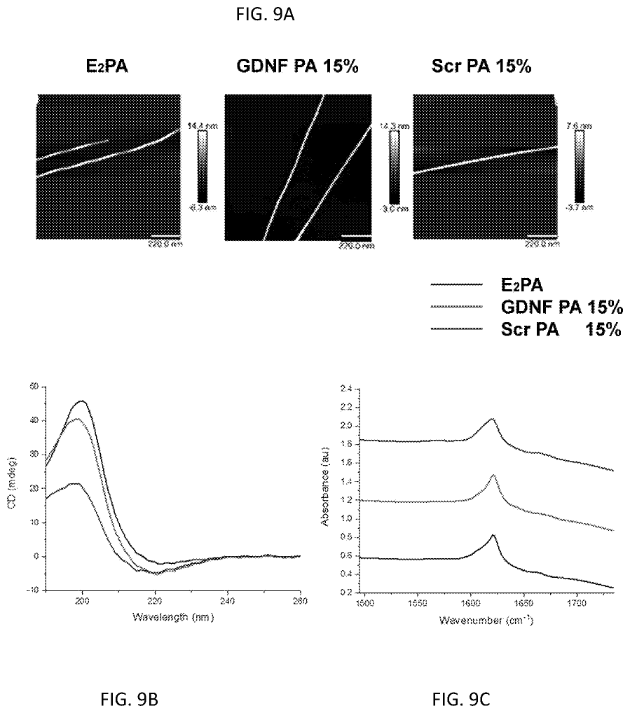

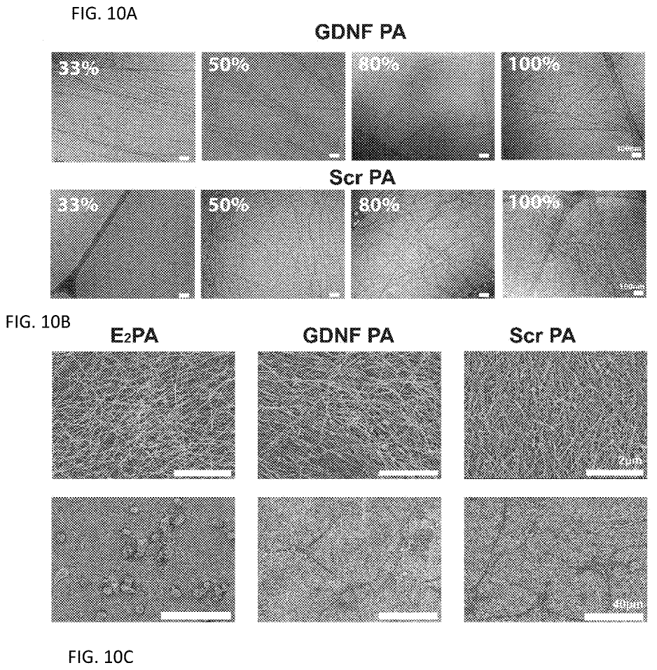

Representative cryogenic transmission electron micrographs (Cryo-TEM) of 15 vol % GDNF PA, 15 vol % Scr PA and E2 PA (FIG. 1c) revealed the formation of ribbon-shaped supramolecular nanofibers. This was confirmed by atomic force microscopy (AFM) imaging, which shows that E2 PA, 15 vol % Scr PA and 15 vol % GDNF PA have very similar twisted nanoribbons (FIG. 9a). The 15 vol % co-assemblies formed longer nanoscale ribbon-like fibers relative to filaments formed by 100% of the Scr and GDNF PAs (compared with FIG. 10a). The 100% GDNF PA showed a wide distribution of nanoribbon lengths and 100% Scr PA showed mostly short ribbons that tended to aggregate (FIG. 10a), whereas the E2 PA backbone alone formed long nanoribbons (FIG. 1c). In addition, the formation of nanofibers was visualized at different volume percentages (80%, 50% or, 33%) between the GDNF PA and Scr PA with the non-bioactive E2 PA (FIG. 10a, Table 2).

To better understand filament formation at the different volume ratios of Scr PA or GDNF PA with E2 PA, synchrotron experiments were performed, including small-angle X-ray scattering (SAXS) and wide-angle X-ray scattering (WAXS). The Scr PA and the GDNF PA were evaluated at 100% (without the diluent E2 PA) as were co-assemblies containing the following volume percent ratios with respect to the E2 PA diluent: 75, 50, 25 and 15 of Scr PA or GDNF PA as well as E2 PA alone. The pure PAs and their co-assemblies all formed fibers. The SAXS profiles show differences in the scattering minima and broadening of the features with greater than 50% of the GDNF PA or Scr PA (FIG. 1d). Above this concentration, these materials exhibited aggregation and polydispersity (consistent with TEM imaging). Wide-angle X-ray scattering (WAXS) was performed to characterize the intermolecular packing within the supramolecular fibers, E2 PA, Scr PA and GDNF PA (FIG. 1e) at the different co-assembly ratios. WAXS revealed a peak consistent with β-sheet hydrogen bonding in 100, 75, 50, 25 and 15 vol % samples. The WAXS diffraction shows reduced crystallinity in the samples with the addition of the GDNF PA or Scr PA. However, the samples do not lose the β-sheet character. To further characterize the β-sheet structure of 15 vol % co-assembly FT-IR and CD spectroscopies were performed (FIGS. 9b and c), which confirmed the SAXS and WAXS data (FIGS. 1d and e).

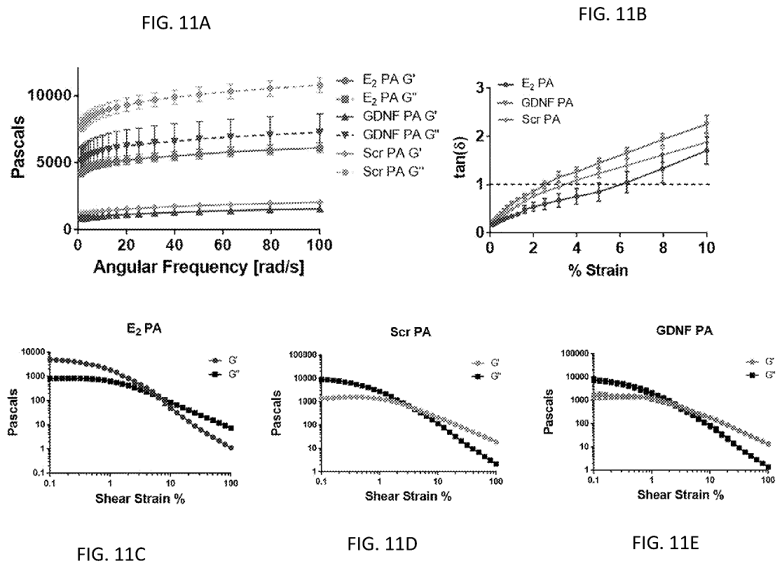

Next, the mechanical properties of gels formed by 15 vol % Scr PA and 15 vol % GDNF PA co-assemblies and E2 PA were evaluated. Rheological analysis indicated that the storage modulus of gels formed by E2 PA was 4.6±0.2 kPa, while co-assemblies with 15 vol % Scr PA and 15 vol % GDNF PA showed storage moduli of 8.7±0.5 kPa and 6.9±1.2 kPA, respectively (FIG. 1f, FIG. 11), which are all in the same range as the moduli of human brain tissue.

Scanning electron microscopy (SEM) was performed to visualize the interaction of neurons with the nanofibers E2, 15 vol % Scr PA and 15 vol % GDNF PA coatings (FIG. 1g) (see Methods section for PA coating procedure). All samples generated homogeneous coatings (FIG. 10b). Low magnification SEM images showed that neurons cultured on the top of E2 PA, 15 vol % Scr PA and 15 vol % GDNF PA formed networks through contacts with other neurons. Neurons seeded on Scr PA and GDNF PA at 15 vol % had a spread morphology compared with cells on E2 PA, which had a rounded morphology (FIG. 10c).

Biological Response of hiPSC-Derived Dopaminergic Neurons Treated with GDNF Mimetic PA

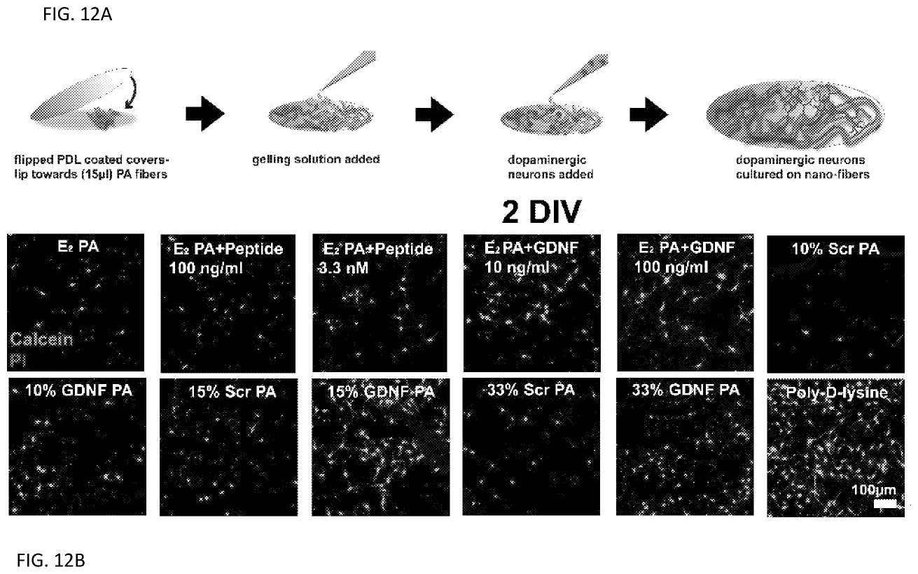

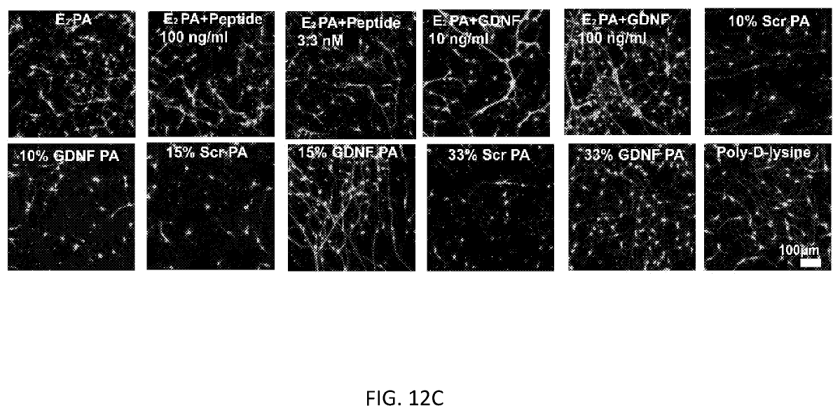

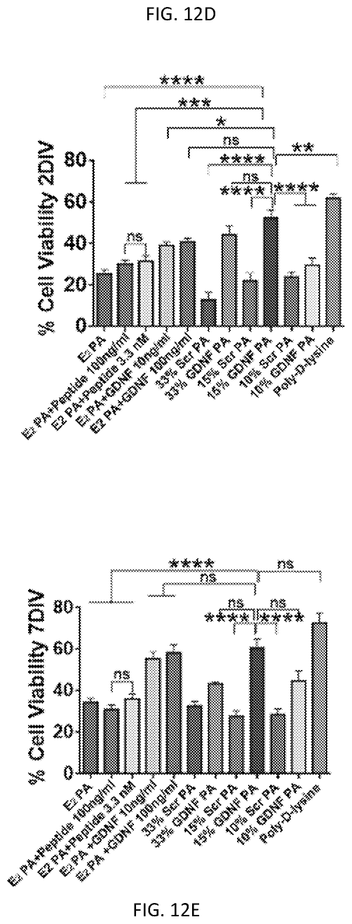

To investigate the best ratio of combination between E2 PA and GDNF PA, coverslips were coated with different ratios of E2 PA/GDNF PA (FIG. 12a). The effect of GDNF PA nanofibers on cell survival was evaluated. Dopaminergic neurons used for this study were derived from human-induced pluripotent cells (hiPSC). The percentage of dopaminergic neurons obtained is approximately 90% (Cellular Dynamics); therefore, in the experiments where a dopaminergic marker is not shown (such as the cell viability assay), it was assumed that 10% of the cells are not dopaminergic neurons.

Cells were seeded on PA coatings at the following co-assembly volume percent ratios: 10, 15 and 33 vol % (for additional information on co-assemblies concentration see Table 2) of GDNF PA with the E2 backbone PA and assessed viability using Calcein AM and propidium iodide staining at 2 days in vitro (DIV) and 7 DIV (-FIGS. 12b and c). These experiments included the following controls: E2 PA coating, E2 PA coating+100 ng/ml peptide (ILKNLSRSR (SEQ ID NO: 1) (92 nM), E2 PA coating+3.3 nM peptide, E2 PA coating+100 ng/ml GDNF protein (3.3 nM) (calculated as homodimer) and E2 PA coating+10 ng/ml GDNF protein. 10 ng/ml and 100 ng/ml of GDNF protein were used based upon reports indicating that RET, the canonical receptor associated with GDNF in dopaminergic neurons, phosphorylates at concentrations in the range of 10 to 100 ng/ml of GDNF protein. At 2 DIV and 7 DIV (FIGS. 12d and e) only the 15 vol % GDNF PA (2 DIV 52.57±3.91%, 7 DIV 60.47±4.42%) co-assembly reached similar percentages of cell viability to the E2 PA coating with 10 (2 DIV 39.27±1.50%, 7 DIV 55.34±3.46%) or 100 ng/ml GDNF (2 DIV 40.87±1.86%, 7 DIV 58.39±3.85%) protein dissolved in the media and to Poly-D-Lysine (PDL) coatings (2 DIV 62.02±2.39%, 7 DIV 72.65±4.79%), which is the most widely used molecule to promote neuronal adhesion³⁹. The other conditions showed significantly lower cell viability (e.g. co-assemblies at 10 vol % (2 DIV 29.67±3.59%, 7 DIV 44.69±5.02%) and 33 vol % (2 DIV 44.40±4.51%, 7 DIV 43.37±0.86%); Scr PA's at 10 vol % (2 DIV 24.02±2.45%, 7 DIV 28.48±2.73%) 15 vol % (2 DIV 22.27±3.75%, 7 DIV 27.81%±2.59) and 33 vol % (2 DIV 13.14±3.5%, 7 DIV 32.80±2.02) vol % and E2 PA (2 DIV 26.60±2.08%, 7 DIV 34.45%±1.88), E2 PA+ Peptide 100 ng/ml (2 DIV 30.35±1.6, 7 DIV 30.96±2.16%) and E2 PA+ Peptide 3.3 nM (2 DIV 31.75±2.5%, 7 DIV 36.16±2.43%). Given this data, the 15 vol % GDNF PA co-assembly (0.78 mM of GDNF PA) is able to mimic the trophic effect of GDNF compared to other co-assembly ratios.

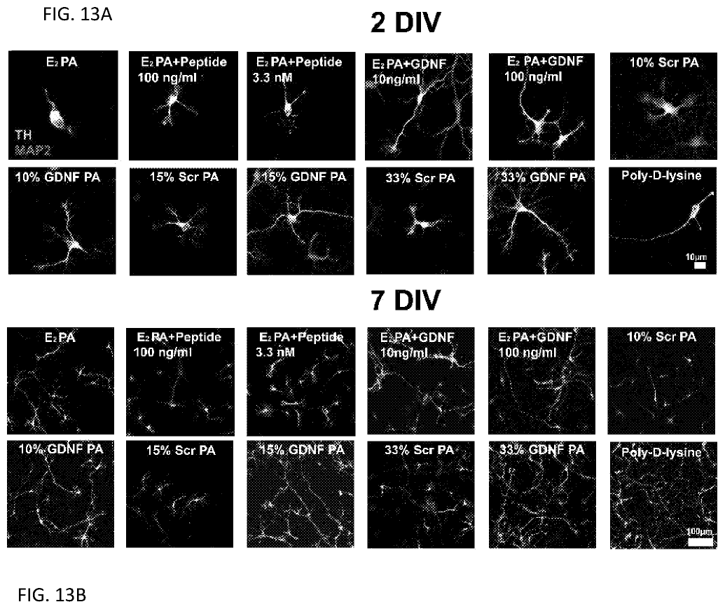

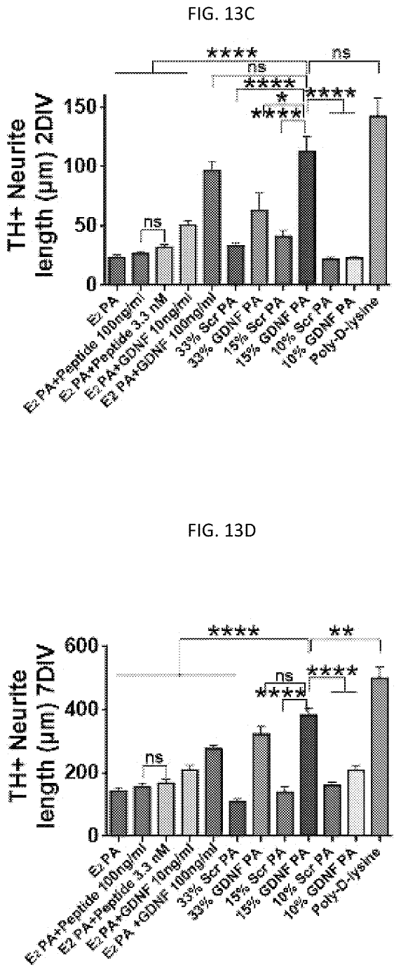

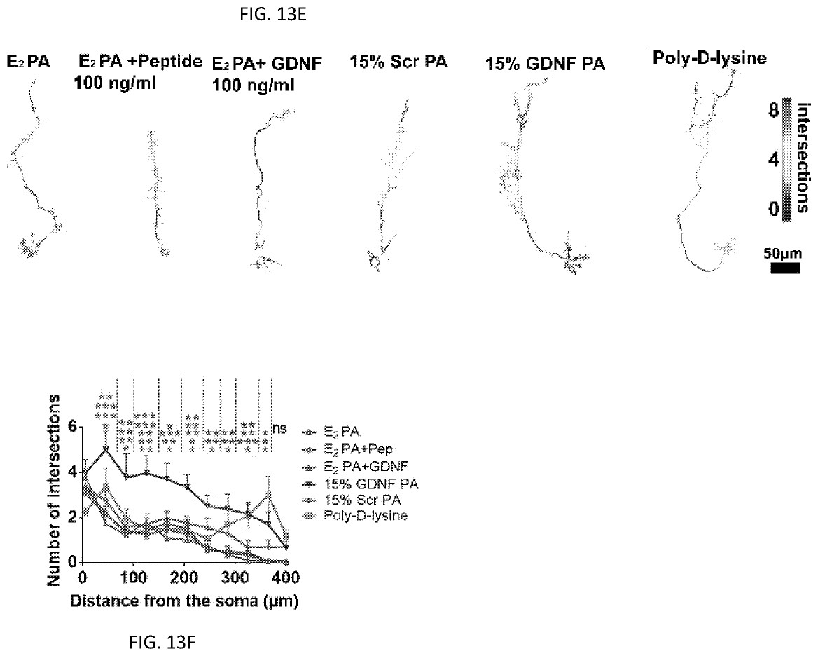

In addition to promoting the survival of dopaminergic neurons, GDNF is also known to enhance neurite outgrowth. To determine whether GDNF PA nanostructures can reproduce this effect, hiPSC-derived dopaminergic neurons were cultured on different PA coatings and dopaminergic neurite outgrowth was measured at 2 DIV and 7 DIV (FIG. 13). Tyrosine hydroxylase (TH) is the rate-limiting enzyme for dopamine synthesis and is commonly used as a marker for dopaminergic neurons, as such anti-TH antibody was used to stain the cells. At 2 DIV the dopaminergic neurons seeded on PDL substrates (142.14±15.73 μm), 15 vol % GDNF PA (112.81±12.75 μm) and E2 PA+100 ng/ml (95.58±8.17 μm) exhibited significantly longer TH+ neurites relative to the other conditions (E2 PA 23.36±2.04 μm, E2+Peptide 100 ng/ml 26.54±1.98 μm or 3.3 nM 32.33±1.95 μm, E2 PA+10 ng/ml 50.66±4.00 μm, GDNF PA vol 33% 62.85±15.13 μm and 10% 22.9±1.47 μm, and Scr PA vol 33% 32.25±3.05 μm 15% 41.08±5.27 μm and 10% 21.90±2.08 μm) (FIG. 13C). After 7 DIV cells on PDL (498.6±34.71 μm) extended significantly longer neurites followed by the 15 vol % GDNF PA co-assembly (382.88±2.77 μm), while the cells exposed to the other conditions displayed shorter TH+ neurites (E2 PA 143.80±10.28 μm, E2+Peptide 100 ng/ml 156.28±11.00 μm or 3.3 nM 167.65±11.72 μm, E2 PA+10 ng/ml 209.42±15.12 μm, GDNF PA vol 33% 322.34±24.28 μm and 10% 207.90±15.94 μm, and Scr PA's vol 33% 109.42±8.25 μm 15% 139.84±16.63 μm and 10% 160.36±10.98 μm) (FIG. 13d).

GDNF promotes the morphological differentiation of ventral midbrain dopaminergic neurons with longer neurites and more complex branching of TH+ fiber. To further characterize the branching complexity, a Sholl analysis was performed. Color-scale images of neurons seeded on different substrates were used to obtain the number of intersections within concentric circles (FIG. 13e). Cells seeded on the 15 vol % GDNF PA co-assembly coating had significantly more TH+ dendritic branches within 300 μm of the cell soma relative to the other conditions (FIG. 13f). From 300 μm to 365 μm, PDL showed similar values to the 15 vol % GDNF PA co-assembly coating and at 400 μm significant differences were not observed among the groups.

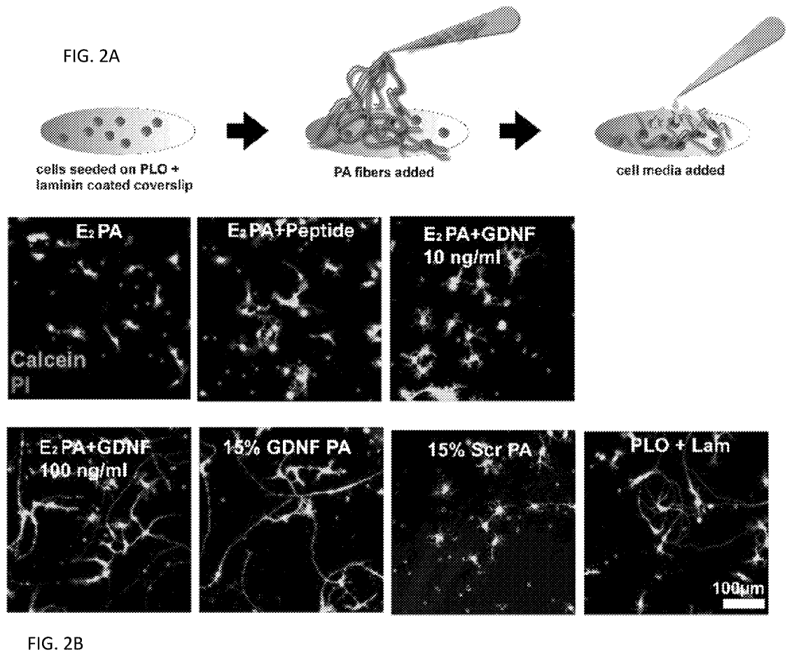

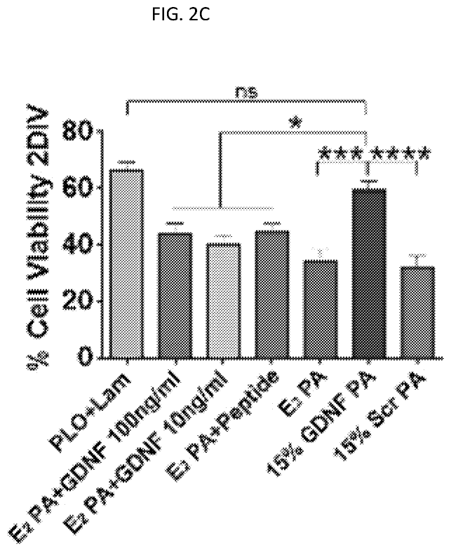

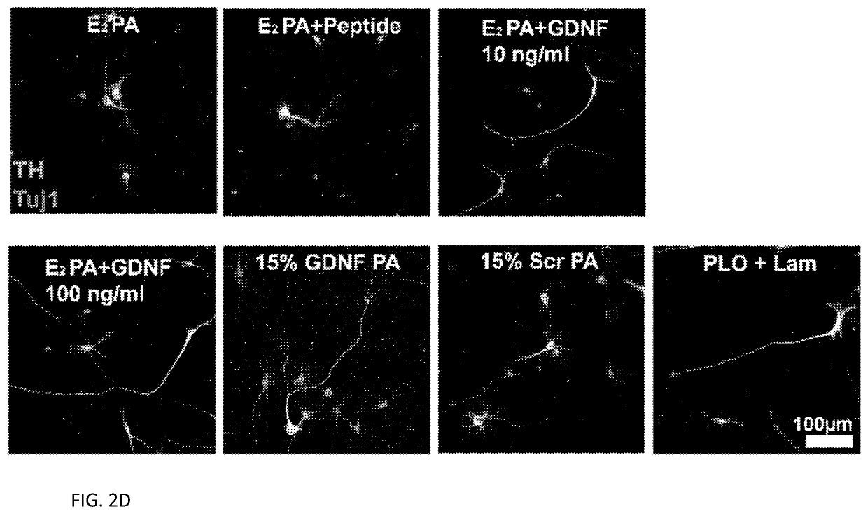

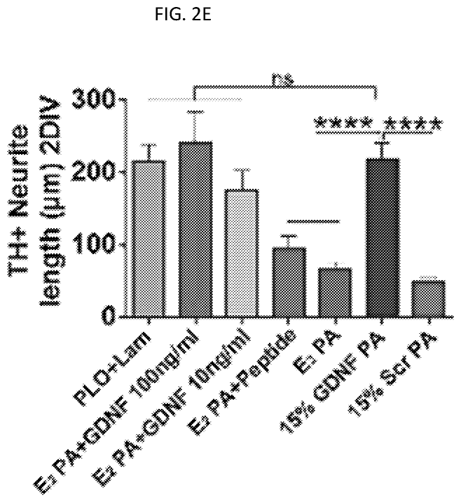

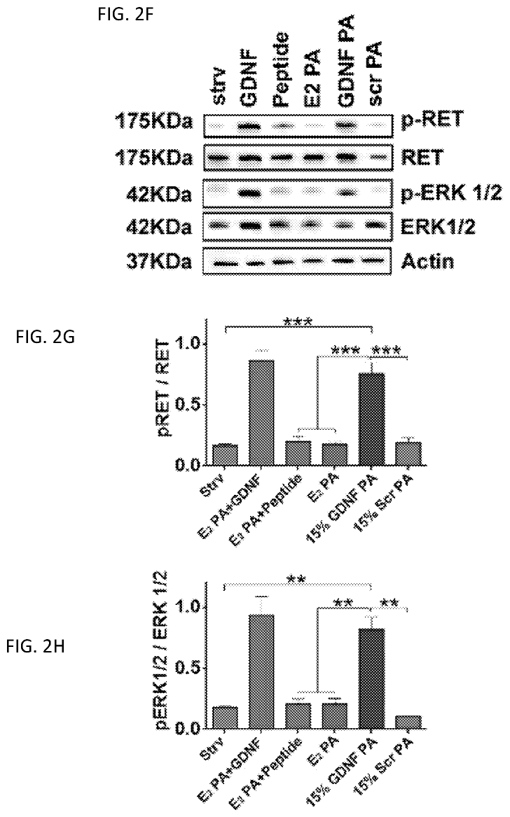

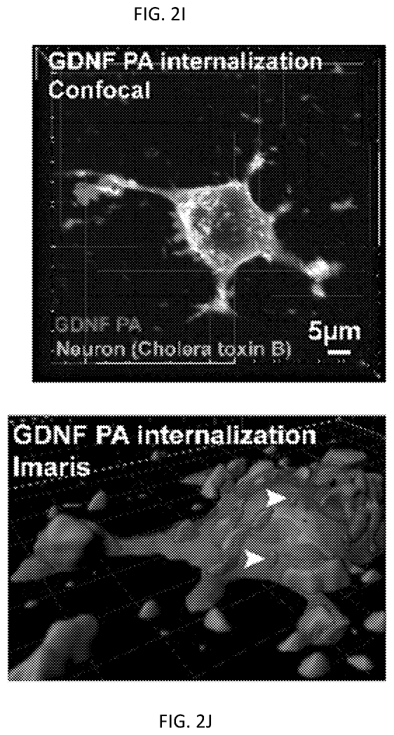

It was hypothesized that at 15 vol % ratio of the two molecules in the supramolecular nanostructures there is a synergistic effect of the bioavailability of the mimetic peptide sequence displayed on the nanostructure surface and the topographical features of the PA nanofibers. To evaluate the biological effects of GDNF PA without the topography influence of nanofibers, cells were seeded on coverslips coated with poly L-ornithine (PLO) and laminin as recommended by the cell manufacturer, and then the PA was added on top (FIG. 2a). Cells were treated with E2 PA, E2 PA+100 ng/ml peptide, E2 PA+10 ng/ml GDNF protein, E2 PA+100 ng/ml GDNF protein, 15 vol % Scr PA, 15 vol % GDNF PA, and PLO. Cell viability and dopaminergic neurite length were evaluated (FIGS. 2b and d). At 2 DIV, the 15 vol % GDNF PA (59.34±3.36%) promoted neuron survival similar to PLO (66.47±2.96%) and significantly higher than the other conditions (E2 PA 34.23±4.19%, E2 PA+Peptide 44.65±2.85%, E2 PA+GDNF 10 ng/ml 40.03±3.53% or 100 ng/ml 43.95±3.68% and 15 vol % Scr PA 31.80±4.44%) (FIG. 2c). In addition, 15 vol % GDNF PA (219.65±21.60 μm) induced comparable neurite length to PLO (215.68±23.7 μm) and E2 PA+GDNF 100 ng/ml (242.45±42.06 μm); the other conditions showed significantly lower lengths (E2 PA 66.36±10.17 μm, E2+Peptide 95.00±17.86 μm and 15 vol % Scr PA 50.16±4.33 μm) (FIG. 2e). The consistency of these results with the PA coating experiments demonstrates that GDNF PA improves cell viability and dopaminergic neurite extension, whether it is utilized as a substrate or as a gel on the top of the cells.

GDNF binds to a GFRα1 receptor to facilitate the binding to the receptor tyrosine kinase (RET) leading to its dimerization and autophosphorylation. Thus, Western Blot experiments were performed to evaluate the phosphorylation of the RET receptor on the following treatments: starvation media (Strv), 100 ng/ml GDNF protein (3.3 nM), 100 ng/ml peptide (92 nM), E2 PA, 15 vol % GDNF PA co-assembly and Scr PA (10.73 mM of PA concentration) (Table 2 for PA concentrations details). Treatments were added after 10 minutes (FIG. 2f) (see methods for details) (see Table 2 for PA concentrations details). The 15 vol % GDNF PA co-assembly induced similar values of phosphorylation of RET and the intracellular ERK1/2 relative to 100 ng/ml of GDNF protein (FIG. 2g-h). After treatment with the bioactive peptide, no effect on the activation of the RET receptor was observed. In contrast, the peptide displayed by the supramolecular nanostructures was found to induce RET phosphorylation. A possible explanation could be the optimal adaptive configurations of bioactive peptides displayed by PA nanofibers, which favor the interaction with cell receptors thus enhancing their bioactivity relative to mimetic peptides.

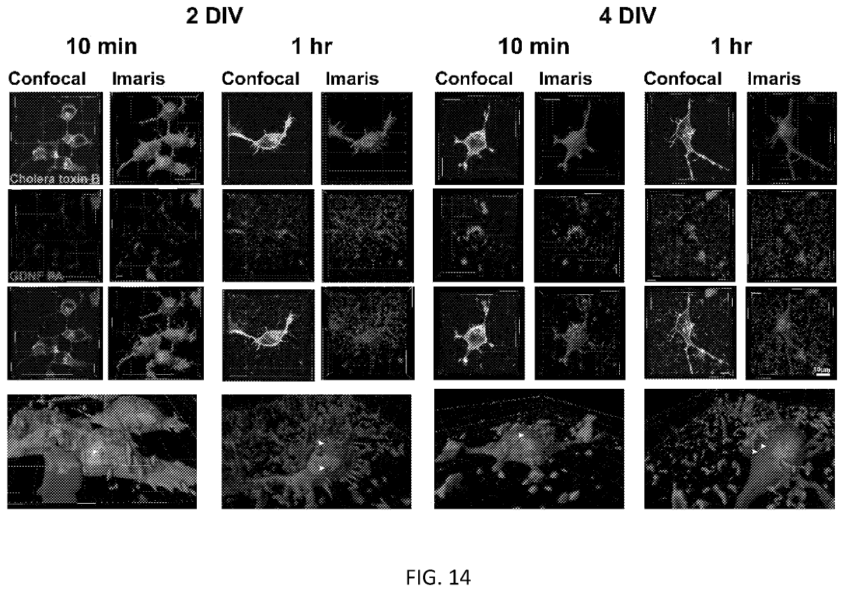

It was next assessed whether neurons could incorporate the PA nanostructures. 2 DIV and 4 DIV neurons were treated with a 15 vol % labeled GDNF PA (C16-VVAAEEK (SEQ ID NO: 16)(TAMRA) ILKNLSRSR (SEQ ID NO: 1) for 10 min and 1 hour, and cholera toxin B was used to label the cell membrane of neurons. In an effort to enhance the visualization of GDNF PA nanostructures inside the neurons, the Imaris Software was used to 3D-render the confocal images. In all the conditions, GDNF PA nanostructures were observed inside the neurons (FIG. 2 i and j, FIG. 14). To monitor the interaction between neurons and GDNF PA in real-time, confocal live imaging was used. Movement of PA nanostructures within the neuron was observed. The experiments revealed that GDNF PA nanostructures are internalized by hiPSC-derived dopaminergic neurons within 10 minutes and 1 hour of exposure.

Pharmacological Inhibition of RET and NCAM Signaling on hiPSC-Derived Dopaminergic Neurons Treated with GNDF and GDNF PA

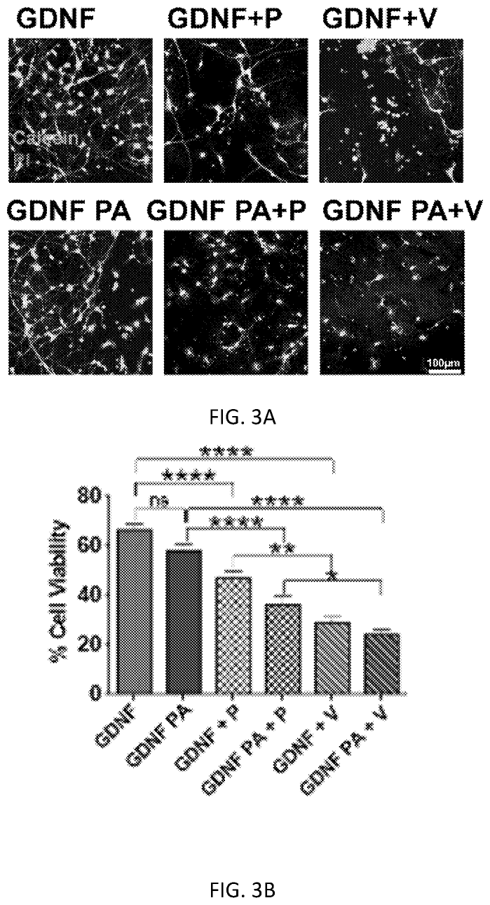

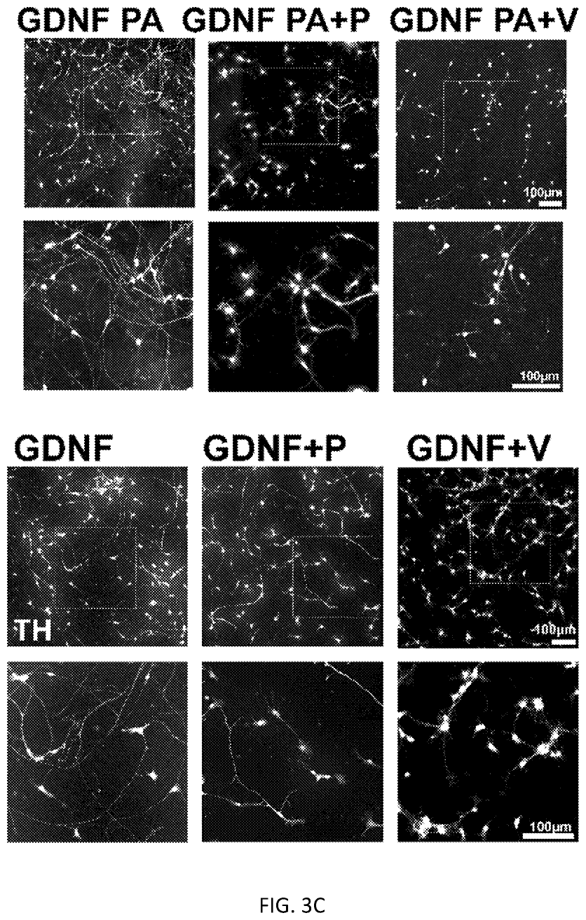

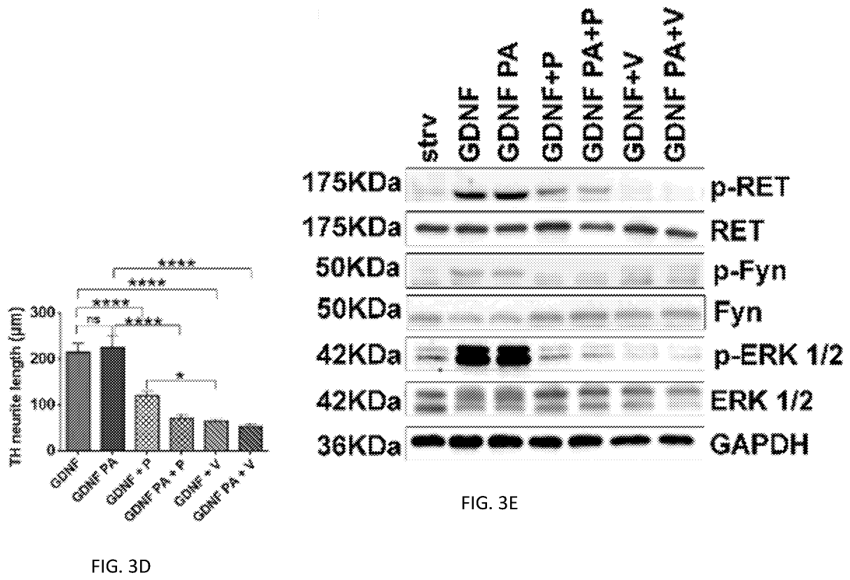

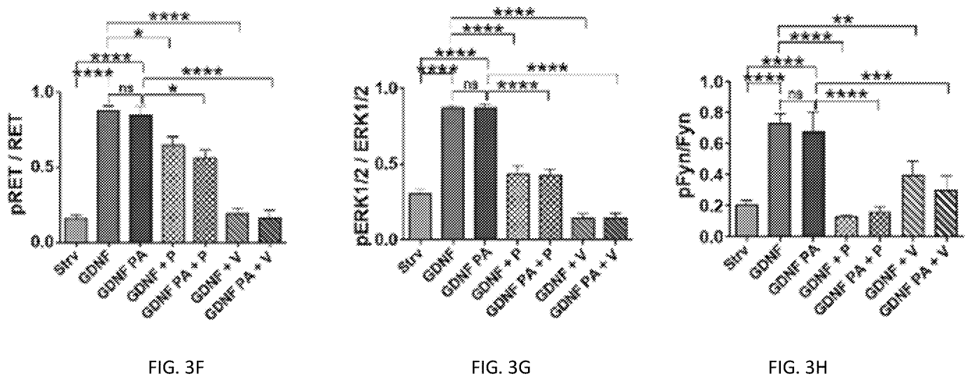

The RET receptor mediates the GDNF effect on cell survival, neuroprotection, and neurodegeneration in dopaminergic neurons. In addition, NCAM plays a role in neurite outgrowth in dopaminergic neurons under GDNF stimulation. To clarify how GDNF PA influences cell survival and dopaminergic neurite growth through RET or NCAM signaling pathways, hiPSC-derived dopaminergic neurons were treated with RET and NCAM inhibitors. RET was inhibited using Vandetanib (V) (1 μM) and NCAM was inhibited with PP2 (P) (1 μM) which blocks the downstream signaling protein of NCAM Fyn. GDNF at 100 ng/ml or 15 vol % GDNF PA was added (gel on the top). Although both inhibitors affected the cell viability, the cells treated with RET inhibitor showed significantly lower survival respect the other conditions (GDNF+P 46.81±2.81%, GDNF+V 29.12±2.21%, GDNF PA+P 36.36±3.46%, GDNF PA+V 24.05±1.99%, GDNF protein 66.47±2.29% and, GDNF PA 57.80±2.96%) (FIGS. 3a and b). In accordance with these results, RET inhibitor reduced the phosphorylated RET and ERK 1/2 (FIG. 3e, f and g). In addition, dopaminergic neurite length was significantly reduced with RET and Fyn inhibitors (GDNF+P 120.00 9.30±μm, GDNF+V 64.34±5.84 μm, GDNF PA+P 70.53±8.60 μm, GDNF PA+V 53.80±5.79 μm) compared to GDNF (214.57±19.32 μm) and GDNF PA (225.82±26.01 μm) (FIG. 2c). The PP2 inhibitor significantly reduced the phosphorylation of Fyn protein (FIGS. 3e and h). The neurite length reduction by RET inhibitor could be an indirect effect of the poor cell viability. These data indicate that the cell viability effect of GDNF PA is mainly mediated by the RET pathway, whereas the NCAM pathway plays a more important role in the enhancement of dopaminergic neurite length.

Transcriptomic Profile and Electrophysiological Maturation of hiPSC-Derived Dopaminergic Neurons Treated with GDNF PA

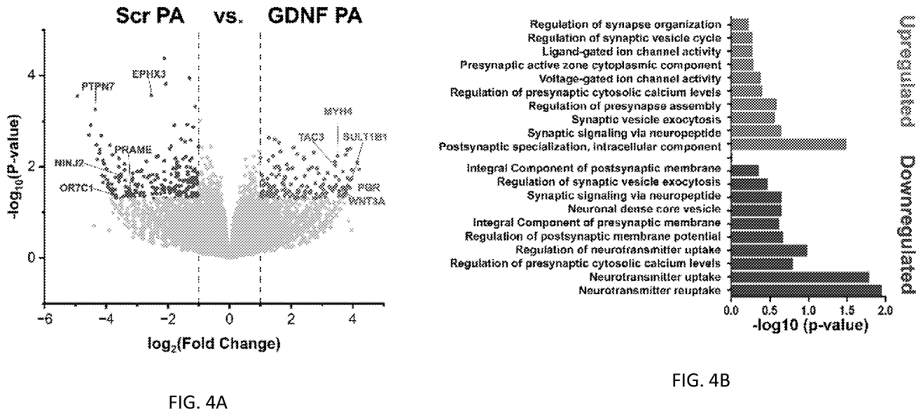

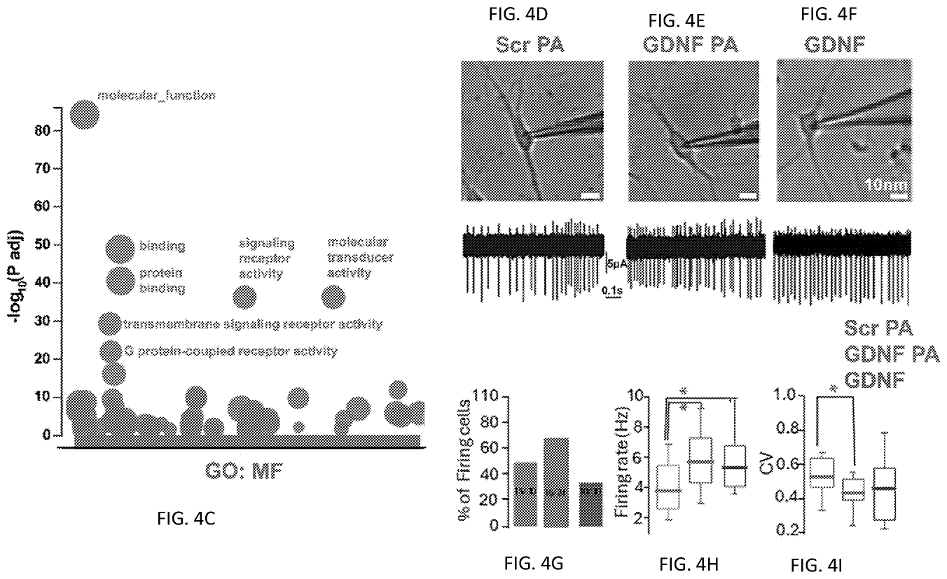

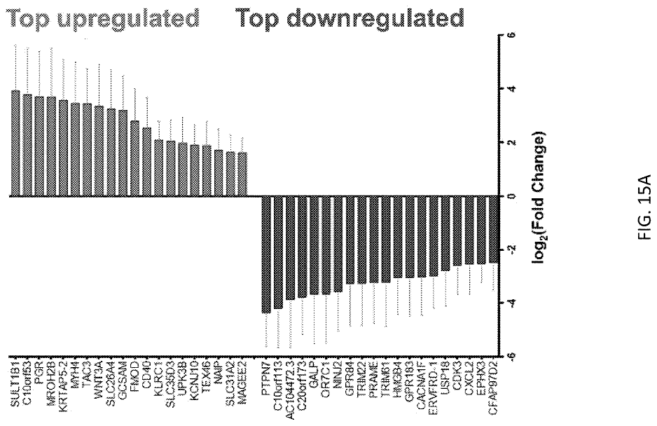

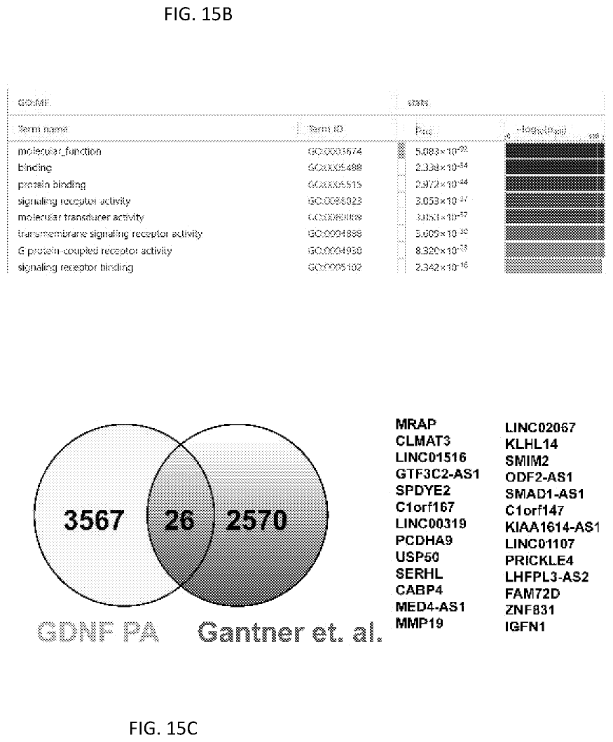

To elucidate the molecular mechanism triggered by GDNF PA nanostructures, RNA sequencing (RNAseq) analysis of hiPSC-derived dopaminergic neurons cultured for 4 DIV on 15 vol % GDNF PA and 15 vol % Scr PA coatings was performed. Analysis of differentially expressed genes (DEGs) comparing 15 vol % GDNF PA (n=4 libraries) vs 15 vol % Scr PA (n=4 libraries) revealed 141 upregulated genes (Fold change cutoff +1, P-value <0.05), whereas 228 genes were downregulated (fold change cutoff −1 and a P-value <0.05) (FIG. 4a). The top 20 most significant up and downregulated transcripts (FIG. 15a) were analyzed. The gene SULT1B1, which encodes for one isoform of sulfotransferase enzymes, was among the most highly expressed genes by hiPSC-derived dopaminergic neurons cultured on 15 vol % GDNF PA. These enzymes are implicated in neuronal cell viability and, interestingly, are downregulated in PD patients. The upregulation of the progesterone receptor gene (PGR) was also observed. This steroid has a neuroprotective effect in PD models in vitro and in vivo. The myosin heavy chain4 (MYH4) also known as MYHC IIb was also upregulated, and this gene plays a critical role in the morphological maturation of dendrites and growth cones and in synaptic function. TAC3 was also upregulated, which encodes the neuropeptide NKB/NK3. This neuropeptide is found in the substantia nigra and plays an important role in the modulation of dopamine release WNT3A was also significantly upregulated which encodes for a glycoprotein that participates in the development of dopaminergic neurons and also has a neuroprotective effect against 6-OHDA.

To evaluate the Gene Ontology (GO) in the synaptic context, an enrichment analysis of the protein-coding DEGs was performed using the gene library SynGO (Koopmans, F. et al. SynGO: An Evidence-Based, Expert-Curated Knowledge Base for the Synapse. Neuron 103, 217-234 (2019)). The postsynaptic specialization GO term was highly enriched (1.47, P-value 0.03375) whereas the neurotransmitter uptake (1.76, P-value 0.01703) and reuptake (1.93, P-value 0.01162) GO terms were significantly downregulated (FIG. 4b) (Table 3). The molecular function (MF) GO terms upregulated by GDNF PA nanostructure were also analyzed through the g:Profiler web (Kolberg, L. et al. G:Profiler-interoperable web service for functional enrichment analysis and gene identifier mapping (2023 update). Nucleic Acids Res. 51, W207-W212 (2023)). (FIG. 4c).

TABLE 3
List of the top 10 up and down-regulated GO terms based on the SynGo library

| | | | | Adjusted | Odds | Combined |
|---|---|---|---|---|---|---|
| | Index | Name | P-value | p-value | ratio | score |
| Upregulated | 1 | Postsynaptic specialization, Intracellular | 0.03375 | 0.5400 | 8.37 | 28.36 |
| | | component (GO: 0099091) CC | | | | |
| | 2 | Synaptic signaling via neuropeptide | 0.2361 | 0.9856 | 4.18 | 6.03 |
| | | (GO: 0099538) BP | | | | |
| | 3 | Synaptic vesicle exocytosis (GO: 0016079) BP | 0.2859 | 0.9856 | 3.25 | 4.07 |
| | 4 | Regulation of presynaptic assembly | 0.2740 | 0.9856 | 2.02 | 2.81 |
| | | (GO: 1905606) BP | | | | |
| | 5 | Regulation of presynaptic cytosolic calcium | 0.4165 | 0.9856 | 1.95 | 1.71 |
| | | levels (GO: 0099509) BP | | | | |
| | 6 | Voltage-gated ion channel activity involved in | 0.4359 | 0.9856 | 1.83 | 1.52 |
| | | regulation of presynaptic membrane potential | | | | |
| | | (GO: 0099508) BP | | | | |
| | 7 | Presynaptic active zone cytoplasmatic | 0.5391 | 0.9856 | 1.33 | 0.82 |
| | | component (GO: 0098831) CC | | | | |
| | 8 | Ligand-gated ion channel activity involved in | 0.5544 | 0.8856 | 1.27 | 0.75 |
| | | regulation of presynaptic membrane potential | | | | |
| | | (GO: 0099507) BP | | | | |
| | 9 | Regulation of synaptic vesicle cycle | 0.5544 | 0.9856 | 1.27 | 0.75 |
| | | (GO: 0098693) BP | | | | |
| | 10 | Regulation of synapse organization | 0.6235 | 0.9976 | 1.04 | 0.49 |
| | | (GO: 0050807) BP | | | | |
| Downregulated | 1 | Neurotransmitter reuptake (GO: 0098810) BP | 0.01162 | 0.1958 | 18.24 | 81.26 |
| | 2 | Neurotransmitter uptake (GO: 0001504) BP | 0.01703 | 0.1958 | 13.68 | 55.72 |
| | 3 | Regulation of neurotransmitter uptake | 0.1647 | 0.7194 | 6.83 | 12.32 |
| | | (GO: 0051580) BP | | | | |
| | 4 | Regulation of presynaptic cytosolic calcium | 0.1081 | 0.7194 | 3.91 | 8.69 |
| | | (GO: 0099509) BP | | | | |
| | 5 | Regulation of postsynaptic membrane | 0.2227 | 0.7194 | 4.55 | 6.84 |
| | | potential (GO: 0060078) BP | | | | |
| | 6 | Synaptic signaling via neuropeptide | 0.2502 | 0.7194 | 3.90 | 5.41 |
| | | (GO: 0099538) BP | | | | |
| | 7 | Neuronal dense core vesicle (GO: 0098992) | 0.2339 | 0.7194 | 2.28 | 3.31 |
| | | CC | | | | |
| | 8 | Integral component of presynaptic membrane | 0.2335 | 0.7194 | 1.48 | 2.15 |
| | | (GO: 0099056) CC | | | | |
| | 9 | Regulation of synaptic vesicle exocytosis | 0.3524 | 0.8635 | 1.66 | 1.73 |
| | | (GO: 2000300) BP | | | | |
| | 10 | Synaptic vesicle proton loading (GO:0097401) | 0.4578 | 0.8635 | 1.71 | 1.33 |
| | | BP | | | | |

Next, whether the GDNF PA coating could induce electrophysiological maturation of hiPSC-derived dopaminergic neurons was investigated. Increasing the firing rate in dopaminergic neurons increases the somatodendritic release of dopamine and produces an increase in the dendritic calcium signal. This increase in calcium signal changes the propensity of dopamine neurons to generate bursts. GDNF protein has been reported to increase the spontaneous firing rate of hiPSC-derived dopaminergic neurons ex vivo. As such, the effect of GDNF PA on the spontaneous firing rate of hiPSC-derived dopaminergic neurons was evaluated. In these experiments, dopaminergic neurons were cultured on 15 vol % Scr PA coating, 15 vol % GDNF PA coating and PDL coatings with 100 ng/ml of GDNF protein (FIG. 4d-f). Cell-attached patch recordings of the spontaneous activity from cells cultured on 15 vol % Scr PA, 15 vol % GDNF PA, and PDL with GDNF protein (100 ng/ml) in the media after 30-35 DIV were measured. The 15 vol % GDNF PA co-assembly coating increased the percentage of cells that presented spontaneous firing activity in contrast to the other conditions (15% GDNF PA coating: 67% (n=24), 15 vol % Scr PA coating: 50% (n=30) and GDNF protein: 33% (n=30)) (FIG. 4g). The neurons seeded on GDNF PA and PDL with GDNF protein presented similar firing rate in contrast with those cells seeded on the Scr PA (15% vol GDNF PA coating: 6.6 Hz (n=16), 15% Scr PA: 3.8 Hz, (n=16), p=0.009) (FIG. 4h). Regarding the coefficient of variation, a significant difference between GDNF PA was found with respect to Scr PA (0.4 vs. 0.5, p=0.02) (FIG. 4i). These results demonstrate that GDNF PA has the same effect of increasing the firing rate of dopaminergic neurons as native GDNF protein.

hiPSC-Derived Dopaminergic Neurons in a Three-Dimensional Culture of GDNF PA

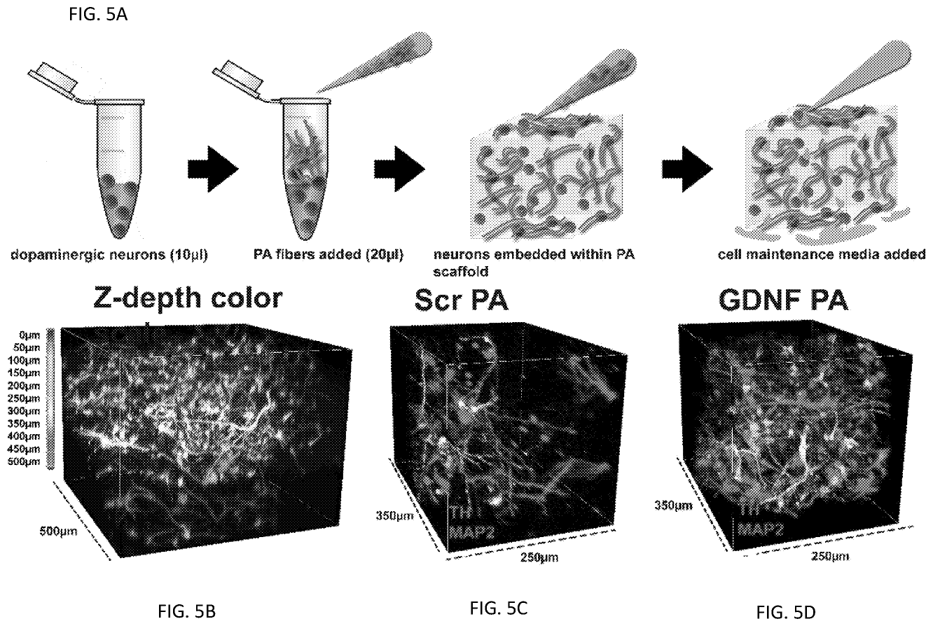

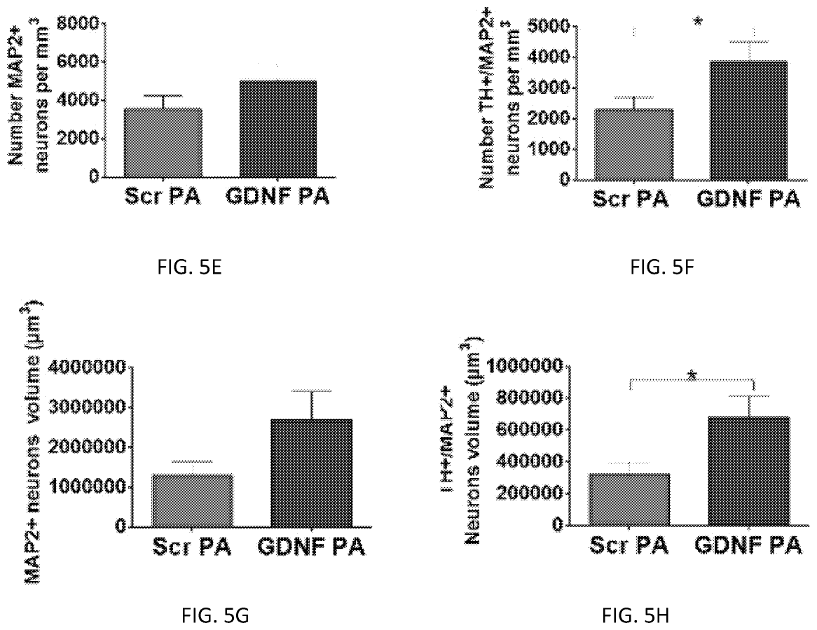

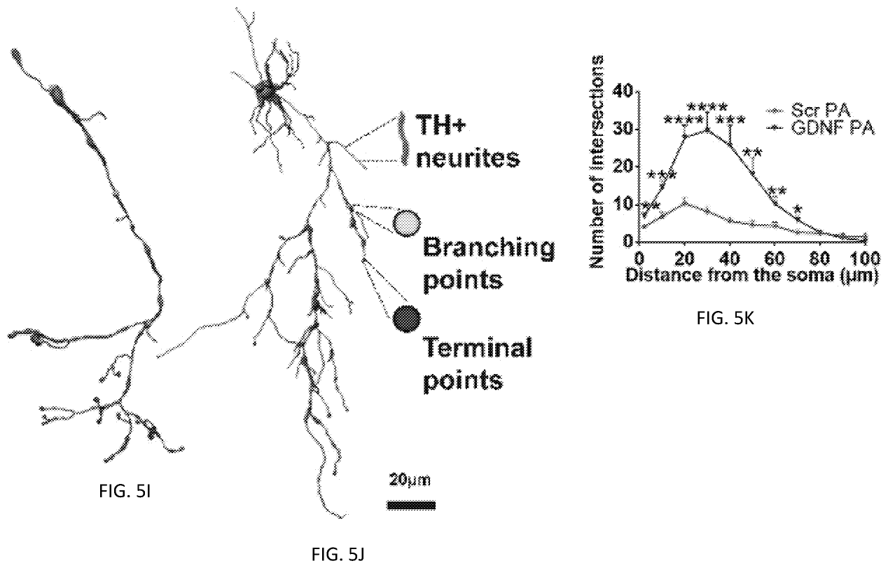

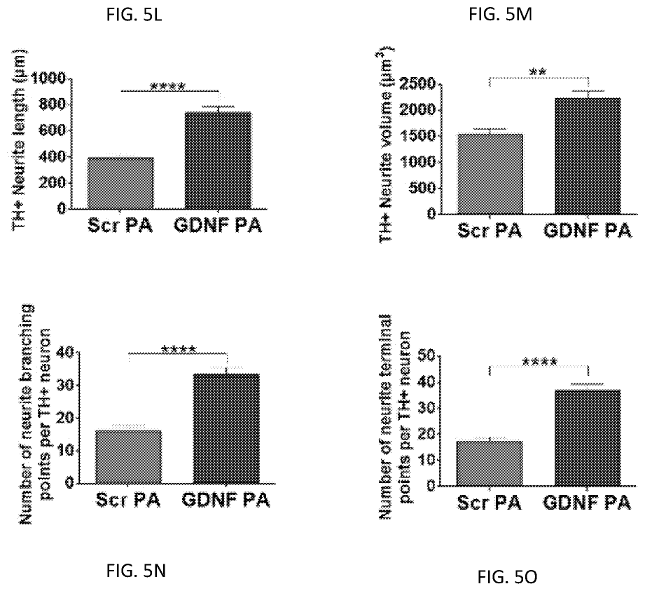

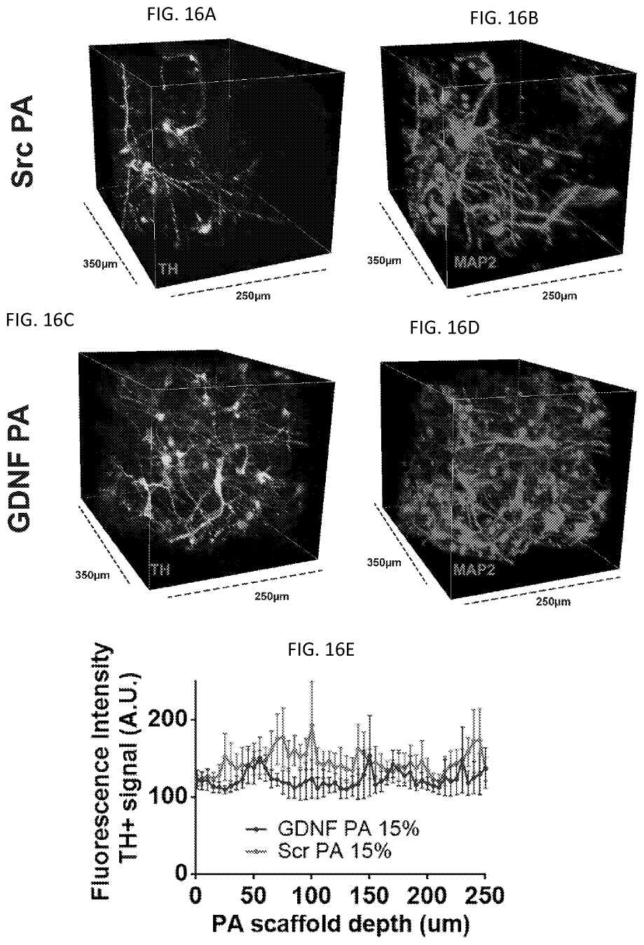

To further develop the bioactive scaffold for cell replacement applications, the interaction of the nanostructures with the cells in three-dimensional cultures was evaluated. Cells were encapsulated by mixing them with the PA solution followed by the addition of cell media (FIG. 5a). Using multiphoton microscopy, it was observed that cells were homogeneously distributed within the scaffolds which are 500 μm thick (see Z-depth color scale in FIG. 5b). To compare the 15 vol % Scr PA and the 15 vol % GDNF PA scaffolds, hiPSC-derived dopaminergic neurons were encapsulated and stained with anti-TH and anti-MAP2 antibodies at 15 DIV (see FIGS. 5c and d for the merged images and FIG. 16 a-d for the separate color channels). The number of TH+ cells (dopaminergic neurons) and MAP2+ cells (matured neurons) per mm³ were quantified. No differences between the conditions were observed for cells stained with the neuronal marker MAP2; however, for TH+/MAP2+ double-stained cells significantly more cells per mm³ in the GDNF PA condition (FIGS. 5e and f) were observed, demonstrating the bioactive effect of GDNF PA respect to the Scr PA. This suggests that the bioactive epitope displayed by the GDNF PA nanofibers induces the survival of the dopaminergic neuronal population, demonstrating the specificity of the GDNF PA. Cell volume, including the cell soma and the neurites, was also calculated, indicative of more mature neurons. A significantly higher volume of TH+/MAP2+ signal were observed in 15 vol % GDNF PA scaffold relative to 15 vol % Scr PA scaffold. (FIGS. 5g and h). Additionally, the distribution of the cells within the gel was evaluated using the TH+ fluorescence signal and it was found that in both conditions the cells were homogeneously distributed (FIG. 16e).

Imaris software was used to reconstruct in three dimensions the morphology of hiPSC-derived dopaminergic neurons embedded in PA scaffolds using multiphoton microscopy images (see methods for details) (FIGS. 5i and j) and several morphometric parameters were measured. Branching complexity was measured by Sholl analysis, and it was found that from 2 μm to 70 μm from the cell soma, the cells encapsulated in the 15 vol % GDNF PA scaffold had significantly more intersections compared to the cells on the 15 vol % Scr PA scaffold (FIG. 5k). The TH+ neurite length was also measured, and it was found that cells embedded in the 15 vol % GDNF PA scaffold had an average value of 744±43.0 μm compared to 394±36.0 μm for cells within the 15 vol % Scr PA (FIG. 5l). The volume of the TH+ fluorescence signal of cells encapsulated in the 15 vol % GDNF PA scaffold was significantly higher (2,239±130.0 μm³) relative to cells encapsulated in 15 vol % Scr PA scaffold (1,541±113.0 μm³) (FIG. 5m). In addition, the number of branching and terminal points per TH+ cell (see the labels in FIG. 5i and 5) were evaluated. The TH+ cells embedded in the 15 vol % GDNF PA scaffold had 33 branching points and 37 terminal points, while the cells in the 15 vol % Scr PA scaffold showed statistically lower values with 16 branching points and 17 terminal points per neuron (FIGS. 5n and o). This data suggests that the 15 vol % GDNF PA can form a suitable 3D scaffold for hiPSC-derived dopaminergic neurons that promotes morphological maturation compared to a scaffold that does not present the bioactive signal (Scr PA).

### GDNF PA Increases Synapse Formation

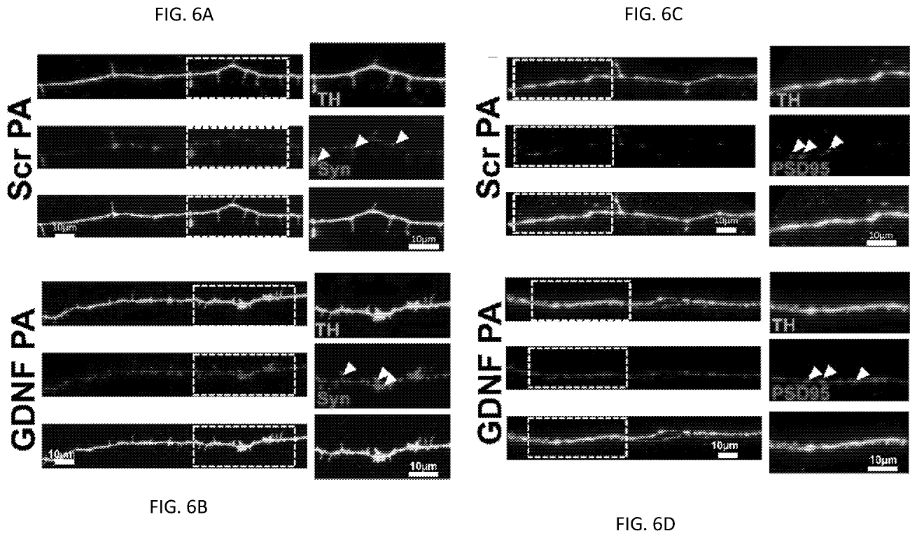

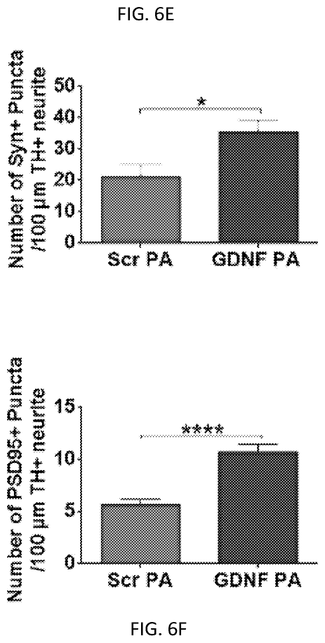

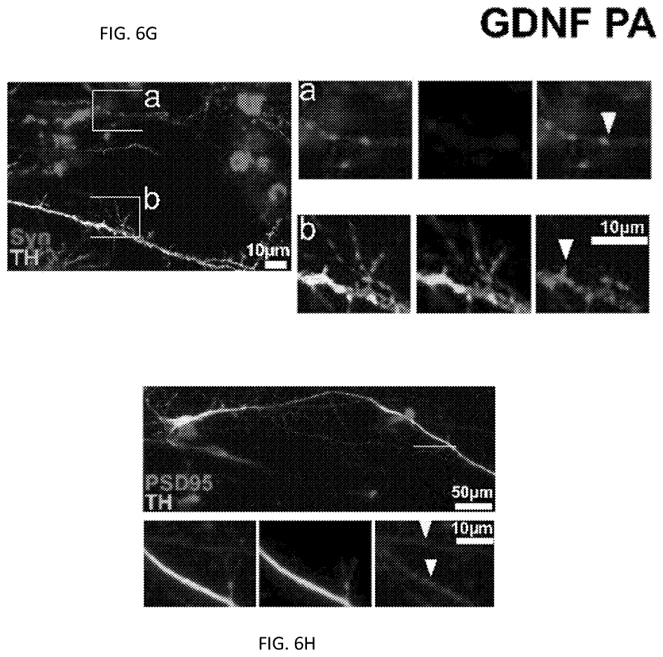

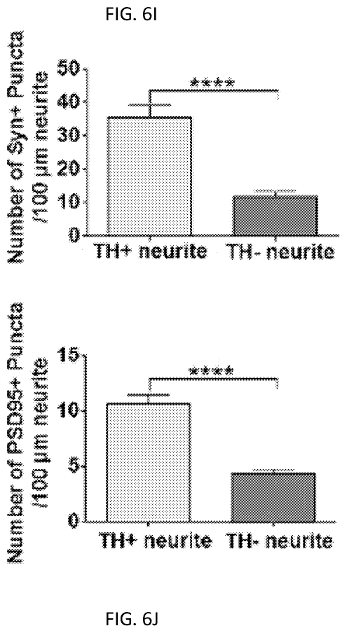

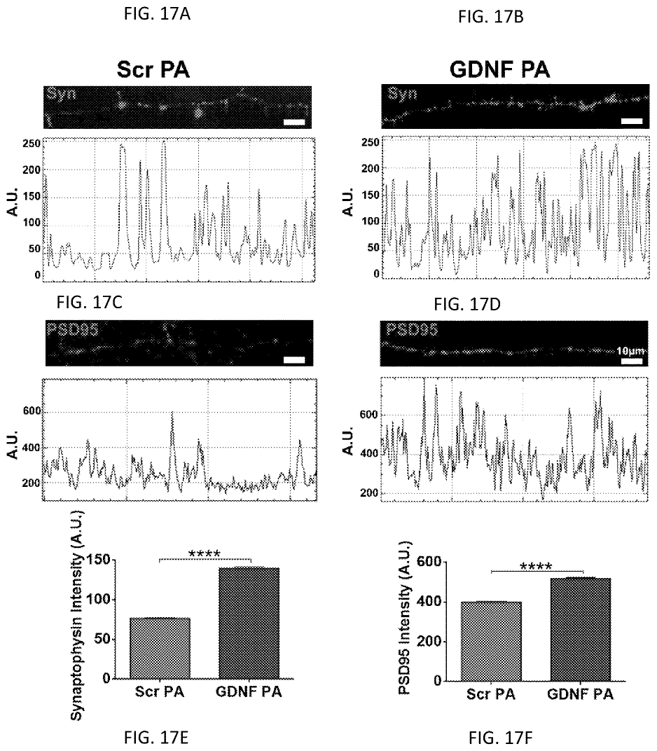

Next, it was investigated whether the GDNF PA scaffold influences the synaptic connections of encapsulated hiPSC-derived dopaminergic neurons which reflects functional maturation. The puncta of the pre-synaptic marker synaptophysin was evaluated in TH+ neurites of cells encapsulated (three-dimensional cultures) in 15 vol % Scr PA scaffold and 15 vol % GDNF PA scaffold (FIGS. 6a and b). Significantly more synaptophysin puncta were observed in cells embedded in 15 vol % GDNF PA scaffold with an average of 35±4.0 per 100 μm of TH+ neurite compared to cells embedded in 15 vol % Scr PA scaffold with an average of 21.2±3.8 per 100 μm of TH+ neurite (FIG. 6e). The midbrain receives glutamatergic inputs which mediate several functions including reinforcement of learning and motor control. Glutamatergic synapses can be examined by the presence of the post-synaptic marker PSD95. Thus, PSD95 puncta were evaluated in neurites of dopaminergic neurons encapsulated in 15 vol % Scr PA and 15 vol % GDNF PA scaffolds (FIGS. 6c and d). Cells embedded in the 15% vol GDNF PA scaffold showed an average of 10.65 puncta per 100 μm of TH+ neurite which was significantly higher than 5.63 of cells embedded in 15 vol % Scr PA (FIG. 6f). To confirm these findings, the fluorescence intensity of the synaptic makers was measured. The fluorescence of synaptophysin and PSD95 in TH+ neurites of cells embedded in 15 vol % GDNF PA scaffold was statistically higher relative to the fluorescence of these markers in TH+ neurites of cells encapsulated in the 15 vol % Scr PA scaffold (FIG. 17).

To determine the specificity of GDNF PA in the enhancement of pre and post synaptic vesicles on dopaminergic neurons, the number of Syn+ puncta on TH+ neurites (35,44±3.78) and TH− neurites (11,7±1.71) was quuantified (FIGS. 6g and i). Additionally, PSD95+ puncta in TH+ neurites (10.65±0.79) respect TH− neurites (4.31±0.41) (FIGS. 6h and j). This data indicates the specificity of the GDNF PA scaffold on the dopaminergic neuron population.

Effect of GDNF PA Nanostructure on the Axonal Extension of Human Midbrain-Like Organoids (hMLOs)

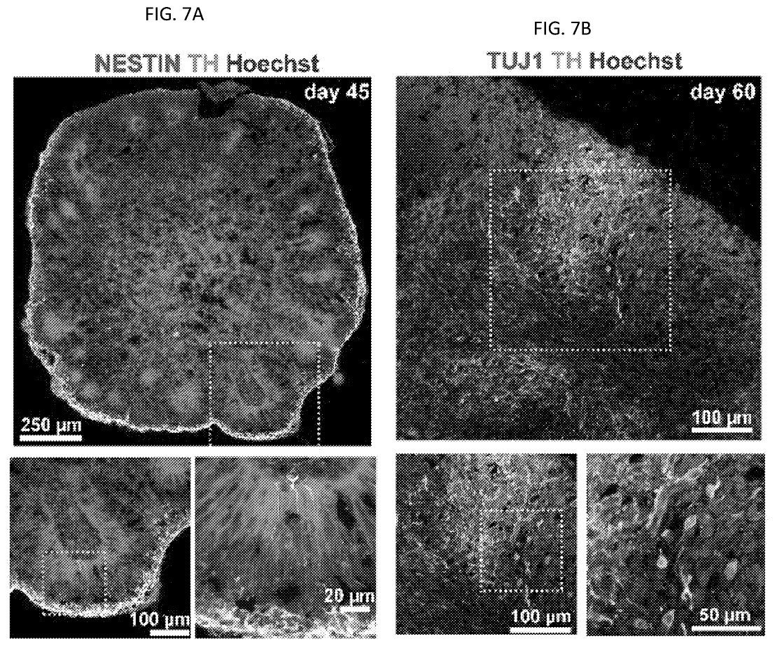

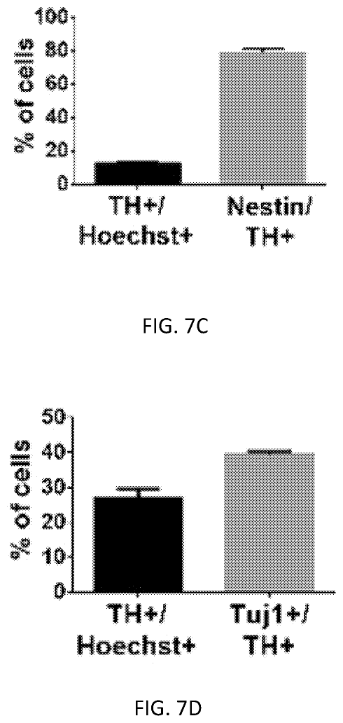

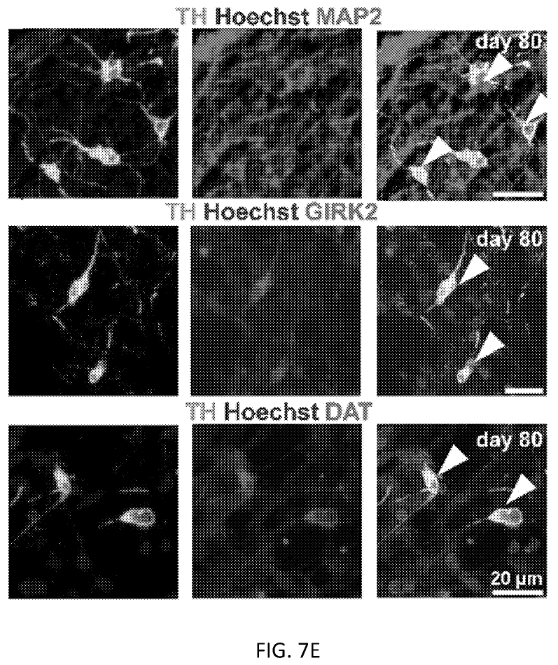

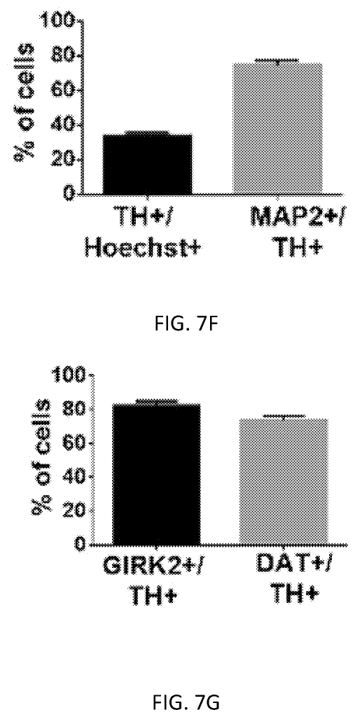

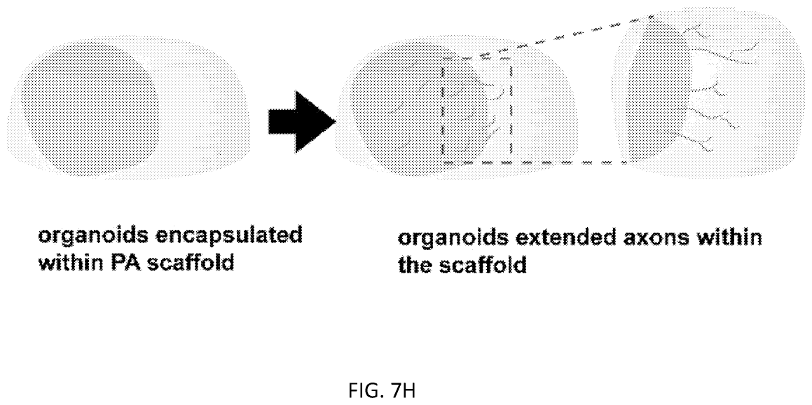

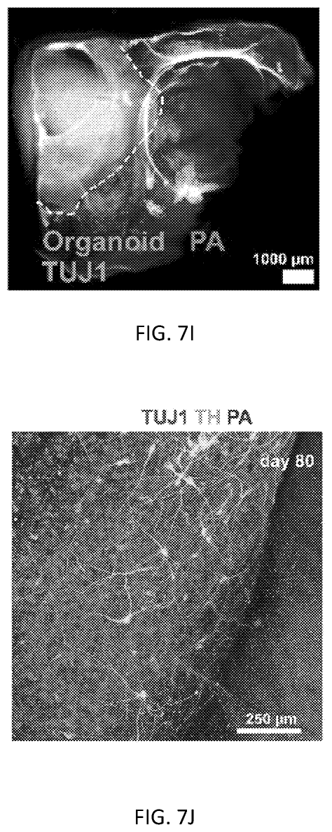

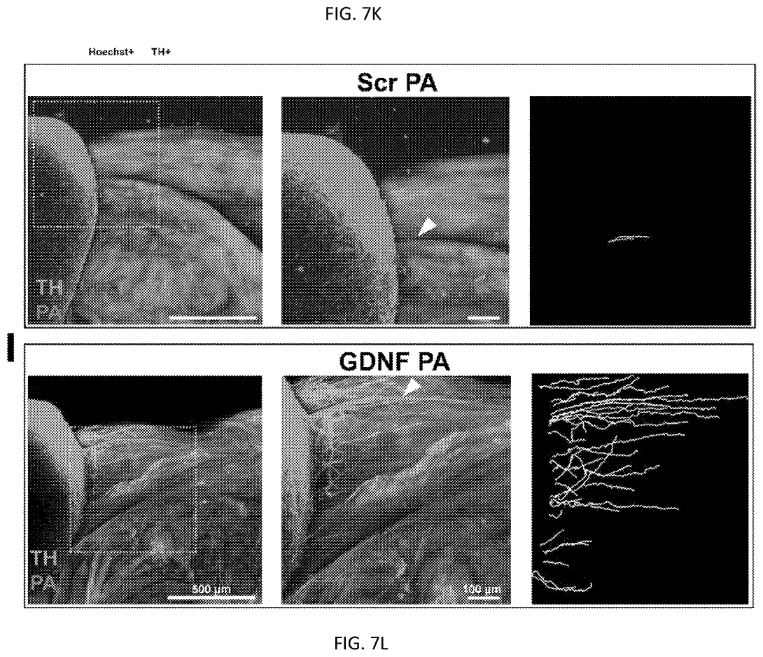

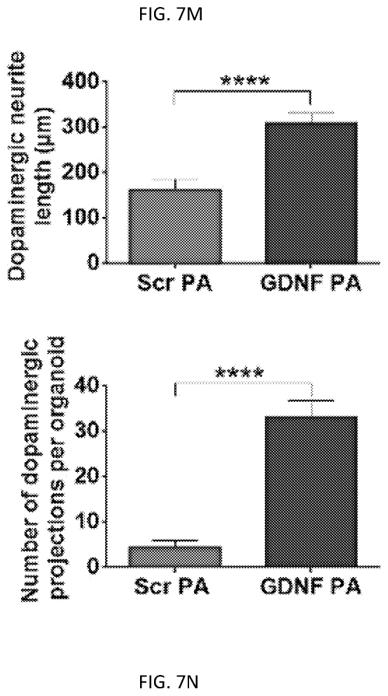

Brain organoids recapitulate several features of human brain development and can be used to evaluate potential therapeutic strategies. Therefore, it was next evaluated whether the GDNF PA scaffold promotes the dopaminergic axonal growth in hMLOs following a protocol reported by Jo et al (Jo, J. et al. Midbrain-like Organoids from Human Pluripotent Stem Cells Contain Functional Dopaminergic and Neuromelanin-Producing Neurons. Cell Stem Cell 19, 248-257 (2016)). The hESC cells used to generate the hMLOs were positive for OCT4, Nanog and SOX2 (data not shown). It was observed that 79% of the cells colocalize for TH and the neural precursor marker nestin (FIGS. 7a and c), whereas only 12.84% were positive for TH at 45 DIV (FIGS. 7a and d). This indicates that at this stage most of the dopaminergic neurons were immature. At 60 DIV 39.6% of neurons were TH+ and Tuj1+ (neuronal marker), and 27.2% of TH neurons did not express Tuj1 (FIGS. 7b and d). After 80 DIV an increase of the dopaminergic population to was observed 34.2% (FIG. 7f). Additionally, the dopaminergic neurons expressed the following maturation markers: 74.8% MAP2, 82.4% GIRK2 (midbrain dopaminergic marker), and 73.8% the dopamine transporter marker DAT (FIG. 7e-g). This data indicates that most of the dopaminergic population reached a mature stage, thus the effect of GDNF PA on 80 DIV organoids was evaluated.

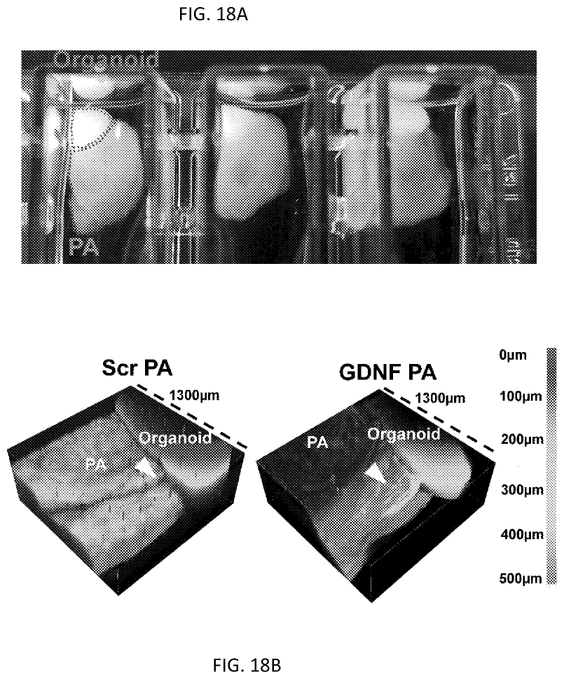

To maintain the organoids in a three-dimensional environment, organoids were encapsulated within 15 vol % Scr PA or 15 vol % GDNF PA (FIG. 7h, i and FIG. 18a) (see methods for details). The organoids were embedded in the PA scaffold for a week, then fixed and stained with TH, Tuj1 and Hoechst dye to visualize the PA (FIG. 7j). Dopaminergic axonal growth from the organoid towards the scaffolds was evaluated. hMLOs embedded in the GDNF PA extended their axons to an average length of 311 μm in contrast to 171 μm in organoids embedded in the Scr PA (FIG. 7k-m). In addition, the number of dopaminergic projections per organoid were counted. An average of 33.2 projections in those embedded in GDNF PA was observed compared to only 4.4 projections when Scr PA was used for encapsulation (the organoids extended their axons at different planes in a range of 500 μm, FIG. 18b). These results are consistent with results for GDNF nanostructures utilized in cultures of hiPSC-derived dopaminergic neurons.

### Neuroprotective Effect of GDNF PA Supramolecular Filaments

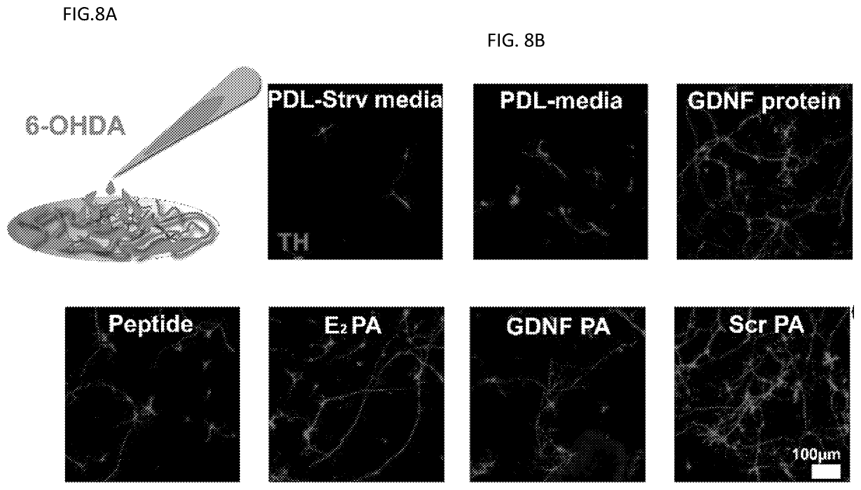

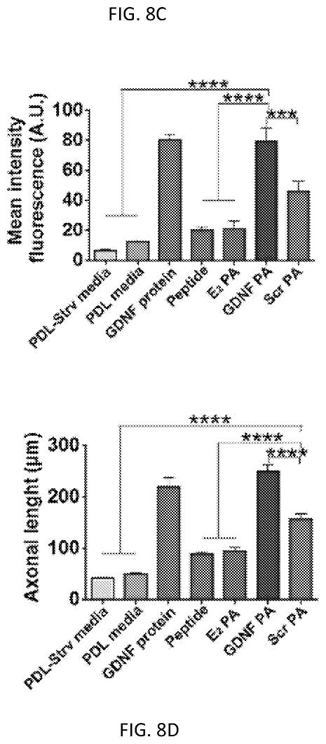

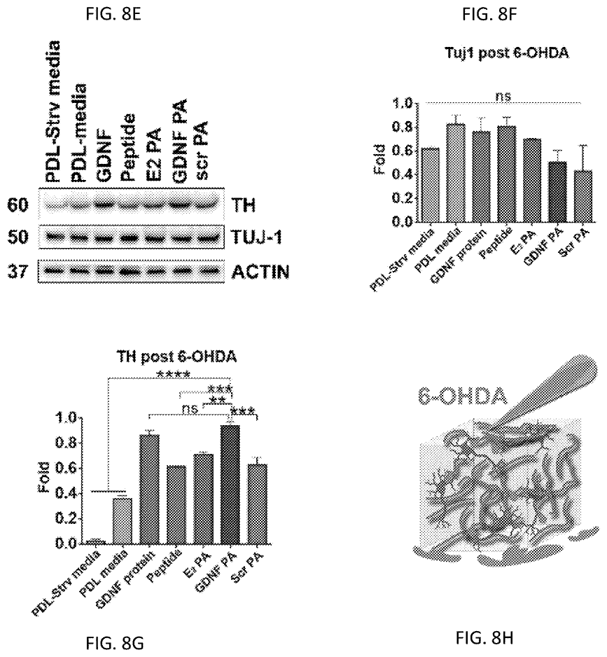

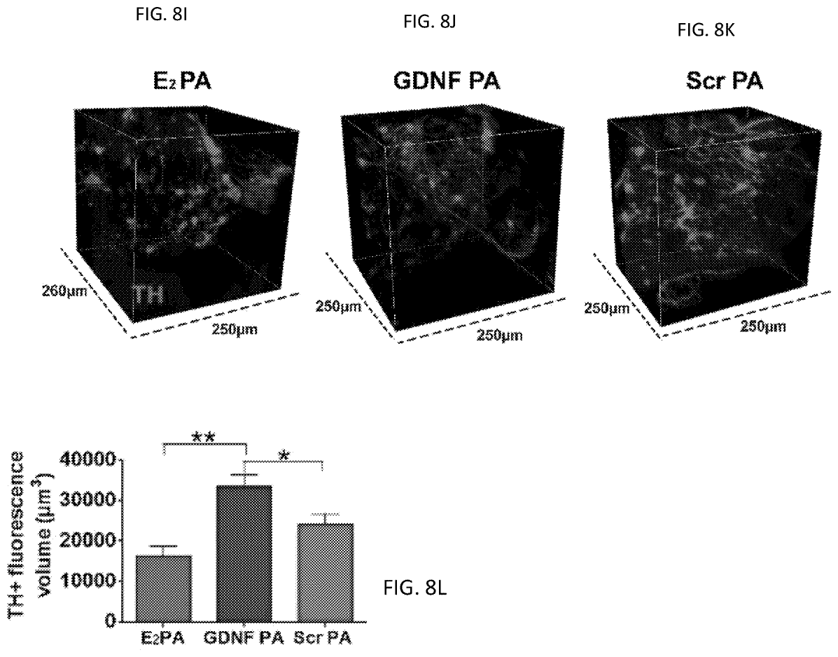

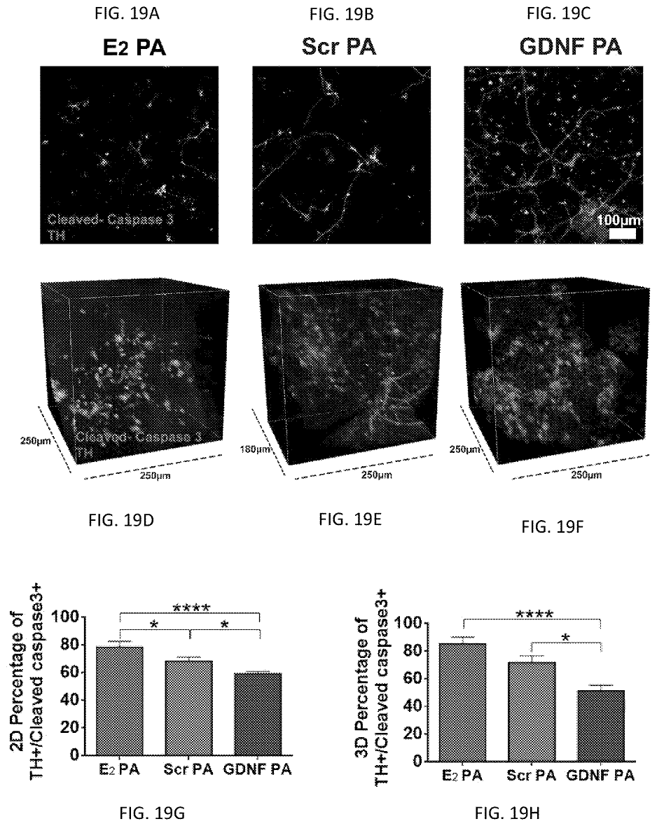

The neurotoxin 6-hydroxydopamine (6-OHDA) is a hydroxylated analogue of dopamine and is commonly used to generate rodent models of PD due to its ability to selectively damage the dopaminergic neurons. It was investigated whether GDNF PA has a neuroprotective effect both in two-dimensional and three-dimensional cell cultures. First, hiPSC-derived dopaminergic neurons were cultured on a PDL substrate with starvation media, PDL substrate with basal media, E2 PA coating with 100 ng/ml GDNF protein, E2 PA coating with 100 ng/ml peptide, E2 PA coating, 15 vol % Scr PA coating and 15 vol % GDNF PA coating. After 15 DIV 100 μM of the neurotoxin 6-OHDA was applied in the media (FIG. 8a) for 1 hour. Immunostaining for TH protein (FIG. 8b) was performed, and the mean intensity fluorescence and the neurite length were measured. Cells seeded on 15 vol % GDNF PA coating reached similar values (79.64±8.92 a.u.) of TH+ mean intensity fluorescence to the cells plated on E2 PA coating with 100 ng/ml GDNF protein (80.56±3.70 a.u.) (FIG. 8c). In addition, the length of dopaminergic neurites of cells seeded on the 15 vol % GDNF PA reached similar values (251.25±12.70 μm) to the cells on the E2 PA coating with 100 ng/ml GDNF protein (219.31±19.11 μm) (FIG. 8d). The other conditions showed significantly lower values for both parameters. The expression of Tuj-1 (neuronal marker) and TH (dopaminergic neuronal marker) proteins was measured by Western blot analysis of two-dimensional cultures at the different conditions (FIG. 8e), and the densitometry showed that there was no statistical difference in the amount of Tuj-1 protein after 6-OHDA treatment (FIG. 8f). In contrast, the TH protein was higher in E2 PA with 100 ng/ml GDNF protein and 15 vol % GDNF PA coating (FIG. 8g), consistent with the immunostaining results. The neuroprotective effect was evaluated in three-dimensional cultures by encapsulating the hiPSC-derived dopaminergic neurons in scaffolds of E2 PA, 15 vol % Scr PA or 15 vol % GDNF PA and then adding 100 μM of the neurotoxin 6-OHDA in the media after 15 DIV (FIG. 8h-k). To estimate the number of dopaminergic somas and neurites after the neurotoxin treatment, the volume generated by TH+ fluorescence staining was evaluated. Cells encapsulated in the 15 vol % GDNF PA scaffold showed significantly higher volume (33583.9±2482.0 μm³) relative to E2 PA (16340.9±2429.5 μm³) and 15 vol % Scr PA (24295.6±2789.4 μm³) scaffolds (FIG. 8l). In addition, double immunostaining for TH and cleaved caspase 3 (a marker of apoptosis) was performed and their co-localization was measured to identify the percentage of dopaminergic neurons that are undergoing apoptosis. Cells seeded on the 15 vol % GDNF PA coating showed 59±2% colocalization compared to 79±4% localization on E2 PA coating and 68. ±3% on the 15 vol % Scr coating (FIG. 19a-c and g). The colocalization of these markers was also evaluated in three-dimensional cultures FIG. 19d-f) and statistically lower colocalization for the 15 vol % GDNF PA scaffold (51.49±4) relative to E2 PA (85.4±5.00) and 15 vol % Scr PA (71.9±4.60) scaffolds (FIG. 19h) was observed. From the similar results obtained in two- and three-dimensional cultures, it is demonstrated that 15 vol % GDNF PA coatings are neuroprotective against the 6-OHDA neurotoxin. This is the first biomaterial that provides this effect without any encapsulated growth factor.

### DISCUSSION

Although cell replacement therapy for Parkinson's disease is very promising, the low survival of transplanted dopaminergic neurons presents a major obstacle to its implementation clinically. The goal of this study was to determine if GDNF-decorated self-assembling nanostructures could be used to overcome this limitation. To achieve this, a peptide mimetic sequence was conjugated with self-assembling nanostructures. Characterization of the supramolecular bioactive filaments revealed a morphology of nanoscale ribbons which can form hydrogels with stiffness values similar to the extracellular matrix of the human brain. An interesting observation from this work has been that optimal bioactivity with the biomimetic peptide is achieved at a characteristic low volume concentration relative to the non-bioactive peptide amphiphile co-assembled to form the supramolecular copolymers. Specifically, the biological effect of different ratio combinations was evaluated, and the co-assembly at 15 vol % for the GDNF PA led to significantly higher bioactivity relative to the other ratios. It was hypothesized that at this vol % ratio the nanofibers can access the RET receptor and trigger the signaling better than lower (10 vol %) or higher (33 vol %) co-assembly ratios of the bioactive PA with non-bioactive E2 PA.

Over the past decade, there has been growing enthusiasm for the use of patient-derived, iPSCc as therapeutic tools. Indeed, hiPSC-derived dopaminergic neurons are being used in a clinical trial with PD patientsHaga clic o pulse aqui para escribir texto. To explore the therapeutic potential of the nanoscaffold described herein, hiPSC-derived dopaminergic neurons were used. GDNF PA nanostructures promoted similar biological effects to the native protein, including cell viability, neurite length, RET and NCAM pathway activation, and neuroprotection. Surprisingly, the GDNF PA enhanced neurite branching compared to the GDNF protein, demonstrating an additive effect of the bioactive signal with the mechanical support of the nanofibers. However, even after eliminating the mechanical support provided by nanofibers by applying PA on top of the cells, the positive effects of GDNF PA on cell viability and dopaminergic neurite length are preserved. This suggests that while ECM-mimicking properties are significant, the primary impact is due to the bioactive components of the GDNF PA nanofiber.

These findings indicate that GDNF PA has comparable efficacy to the substrate recommended by the cell provider in promoting cell viability and dopaminergic neurite extension. However, unlike PLO, GDNF PA maintains its efficacy in three-dimensional cultures, including those shown in FIG. 5 and midbrain-like organoids in FIG. 7. This characteristic is essential for the potential in vivo application of this matrix to support cell viability, which is not achievable with PLO or other substrates like PDL and laminin.

Interestingly, the RNA-seq data was robustly correlated with the morphological and functional effects of the GDNF nanostructure on hiPSC-derived dopaminergic neurons. The transcript with the highest upregulation value was SULT1B1, which encodes a sulfotransferase, an enzyme that sulfonates dopamine to inactive dopamine sulfate. This is essential in cell survival mechanisms since free dopamine can lead to cell death through oxidative stress and autoxidation. Indeed, the upregulation of SULT1B1 could provide a molecular explanation for the enhanced cell viability induced by GDNF PA coatings. In addition, the GDNF nanostructure upregulated WNT3a and the progesterone receptor gene (PGR), both of which protect dopaminergic neurons against toxins. Additionally, progesterone also promotes the differentiation of mouse embryonic stem cells into dopaminergic neurons. Overall, these results explain the neuroprotective effect of the GDNF PA scaffolds against the 6-OHDA toxin. Interestingly, more dopaminergic neurons were found in GDNF PA scaffolds relative to Scr PA scaffolds. Another significantly upregulated gene was MYH4 (also known as MYHC IIb), whose protein product promotes dendritic spine maturation through the remodeling of actin filaments. This upregulation may assist in synapse formation in neurons embedded within GDNF PA scaffolds. Lastly, significant upregulation of TAC3, which encodes the neuropeptide NK3, was also observed. Here, it is proposed that the electrophysiological maturation observed under GDNF PA treatment is attributable to the normal maturation of the intrinsic neuron. Overall, RNA data indicate that GDNF PA enhances the viability and accelerates the maturation of human dopaminergic neurons.

Results of dopaminergic neurons embedded within GDNF PA demonstrated that this bioactive scaffold enhances maturation in a three-dimensional environment that mimics natural ECM compared to traditional two-dimensional cultures. This distinguishes the GDNF PA nanostructures from other general matrices used to culture neurons in a two-dimensional environment such as PDL, PLO, laminin, or gelatine. Recently it has been demonstrated that brain organoids recapitulate the development of the human brain preserving its genetic background in contrast to neurons obtained from a differentiation process in 2D. Therefore, brain organoids are emerging as an essential model to test therapeutic strategies for neurological diseases. To evaluate if the bioactive nanostructure described in this work exerts a biological effect in this biological platform, 80-day hMLOs were generated from hESC. The organoids embedded in the GDNF PA extended more and longer dopaminergic axons compared to those encapsulated in the Scr PA. These results also confirmed the effects observed in cultures of hiPSC-derived dopaminergic neurons.

Cell replacement therapies using human dopaminergic neurons derived from hESC or hiPSCs have demonstrated motor behavior improvement in rodent and monkey models of PD, respectively. However, several obstacles remain to moving these studies into the clinic. One of them is the very low cell survival reported in those studies, ranging between 2.6 to 6 percent. The GDNF biomimetic nanostructure described here improved the viability of human dopaminergic neurons. This is in contrast to previous works in which the native protein was encapsulated in a nanofiber matrix and then released into brain tissue. However, this strategy is well known to have several limitations, such as a short half-life of the protein in tissue. The GDNF PA nanostructure has the potential to avoid these problems, since the signal is a part of the scaffold. In addition, the supramolecular systems developed herein have enormous potential for tunability with minor changes in the part of the peptide sequence that controls morphology, fiber stiffness, and dynamics which should include diffusion of small clusters of bioactive molecules but without affecting the bioactive sequences that activate receptors. This represents a significant advantage over other alternatives, as GDNF PA can be tailored to achieve the mechanical properties needed for the desired diffusion in vivo. Further in vivo studies are needed to evaluate the potential effect of GDNF PA on the cell viability of transplanted human dopaminergic neurons.

### Example 2

The following exemplary methods were used in the experiments described herein.

### Peptide Synthesis

Peptide amphiphiles (PA) molecules were synthesized on solid support with Rink amide resin via Fmoc-based chemistry on a CEM Liberty Blue automated microwave peptide synthesizer. The automated coupling reactions were performed using Fmoc-protected amino acid (4 eq.), DIC (4 eq.), and Oxyma pure (8 eq.). Fmoc deprotection was completed using 4-methylpiperidine (20% in DMF). The resin-bound peptides were cleaved using a mixture of TFA (95%), water (2.5%), and TIPS (2.5%). The peptides were precipitated with cold ether followed by HPLC purification using a Waters Prep 150 LC system or a Shimadzu Prominence system in water/acetonitrile gradients containing 0.1% NH₄OH (or 0.1% TFA), both equipped with Phenomenex Gemini C18 columns. Target compounds were identified from fractions using direct injection electrospray ionization mass spectroscopy on an Agilent 6520 Q-TOF system. Evaporation of volatile solvents followed by lyophilization resulted in white powders.

### NMR Spectroscopy

Proton nuclear magnetic resonance (¹H) spectra were recorded on a Bruker Neo 600 MHz system with a QCI-F cryoprobe (sensitivity: ¹H=5000, ¹³C=800). Carbon nuclear magnetic resonance (13C) spectra were recorded on a Bruker Avance III 500 MHz with a DCH CryoProbe (sensitivity: ¹H=1800, ¹³C=1400). NMR spectra were all recorded at 25° C. using DMF-d₇ as solvent with 5% (v/v) D₂O. Chemical shifts are reported in parts per million (ppm) in relation to solvent peak DMF-d₇ (¹H: 8.03, 2.90, 2.75 ppm; ¹³C: 163.1, 34.9, 29.8 ppm).

E2 PA (C16-V2A2E2 (SEQ ID NO: 10)). NMR spectra confirmed the structure of the PA. Purity was verified by LCMS.

GDNF PA (C16-V2A2E2ILKNLSRSR (SEQ ID NO: 17)). Purity analysis and structural characterization were performed. ¹H-NMR (600 MHz, DMF-d₇): 6=4.61-4.57 (m, 1H), 4.33-4.28 (m, 2H), 4.26-4.24 (m, 1H), 4.20-4.17 (m, 2H), 4.14-4.10 (m, 1H), 4.08-3.90 (m, 8H), 3.88-3.78 (m, 4H), 3.36-3.29 (m, 4H), 3.09-3.03 (m, 3H), 2.94-2.90 (m, 2H, note: overlap with solvent peak), 2.81-2.68 (m, 3H, note: overlap with solvent peak), 2.54-2.37 (m, 6H), 2.18-1.56 (m, 26H), 1.53 (d, 3H, J=7.2 Hz, Ala-β), 1.51-1.45 (m, 4H), 1.34-1.22 (m, 24H), 1.10 (d, 3H, J=6.7 Hz, Val-γ), 1.04-1.02 (m, 6H, 2×Val-γ), 0.95-0.85 (m, 24H, Ile-6, Ile-γ′, 4×Leu—6, Pal-ε, Val-γ); ¹³C-NMR (125 MHz, DMF-d₇): 6=176.7, 176.5, 176.4, 176.1, 175.5, 175.5, 175.5, 175.4, 175.3, 175.2, 175.0, 174.4, 174.2, 174.2, 173.9, 173.0, 172.1, 171.8, 170.5, 157.4 (imine), 157.4 (imine), 64.3-62.5 (several carbons), 61.5, 61.0, 58.6, 57.3-56.6 (several carbons), 54.6 (2C), 53.3, 53.0-52.5 (several carbons), 41.1, 40.9, 39.6, 39.3 (2C), 39.19, 36.1-34.9 (several peaks, overlap with solvent peak), 31.9, 30.6-24.9 (multiple carbons, overlap with solvent peak), 24.6, 24.3, 23.1, 23.0, 22.5, 22.4, 21.4, 21.1, 20.8, 20.2, 18.8-15.6 (multiple carbons), 14.7, 13.7, 10.9. (note: several carbon peaks are undetectable due to insufficient signal-to-noise). HRMS (ESI/Q-ToF): calcd for C₈₈H₁₆₀N₂₄O₂₃, 1921.2088; found 1921.2082.

Scr PA (C16-V2A2E2LRNKSRILS (SEQ ID NO: 18)). Purity analysis and structural characterization were performed. ¹H-NMR (600 MHz, DMF-d₇): 6=4.58-4.61 (m, 1H), 4.31-4.34 (m, 1H), 4.20-4.26 (m, 3H), 4.00-4.14 (m, 9H), 3.91-3.94 (m, 3H), 3.82-3.86 (m, 2H, overlap with HDO-peak), 3.41-3.30 (m, 4H), 3.09-2.99 (m, 3H), 2.94-2.93 (m, 2H, overlap with solvent peak), 2.81-2.77 (m, 3H), 2.48-2.33 (m, 6H), 2.17-1.48 (m, 33H), 1.22-1.34 (m, 24H), 1.09 (d, 3H, J=6.7 Hz), 1.03 (d, 3H, J=6.5 Hz), 1.01 (d, 3H, J=6.8 Hz), 0.84-0.95 (m, 24H). HRMS (ESI/Q-ToF): calcd for C₈₈H₁₆₀N₂₄O₂₃, 1921.2088; found 1921.2102.

Peptide (ILKNLSRSR (SEQ ID NO: 1). Purity analysis and structural characterization were performed. ¹H-NMR (600 MHz, DMF-d₇): δ=4.72-4.68 (m, 1H, Asn-α), 4.52 (dd, 1H, J=9.6, 5.4 Hz, Leu-α), 4.43-4.34 (m, 6H, 2×Arg-α, Leu-α, Lys-α, 2×Ser-α), 4.07 (d, 1H, J=5.7 Hz, Ile-α), 3.89 (dd, 1H, J=11.3, 6.3 Hz, Ser-β), 3.86 (dd, 1H, J=11.1, 6.0 Hz, Ser-β), 3.81 (dd, 1H, J=11.3, 5.1 Hz, Ser-β), 3.76 (dd, 1H, J=11.1, 5.4 Hz, Ser-β), 3.33-3.25 (m, 4H, 2×Arg-δ), 3.03 (t, 2H, J=7.6 Hz, Lys-ε), 2.84-2.76 (m, 2H, Asn-β), 2.08-2.01 (m, 1H, Ile-β), 2.00-1.92 (m, 2H, Arg-β), 1.84-1.57 (m, 17H, Arg-β, 2×Arg-γ, Ile-γ, Leu-β, 2×Leu-γ, Lys-β, Lys-γ, Lys-δ), 1.55-1.42 (m, 2H, Leu-β), 1.36-1.24 (m, 1H, Ile-γ), 1.05 (d, 3H, J=6.9 Hz, Ile-γ′), 0.92-0.85 (m, 15H, Ile-6, 4×Leu-δ); ¹³C-NMR (125 MHz, DMF-d7): δ=174.2 (carbonyl), 173.5(carbonyl), 173.1(carbonyl), 172.6(carbonyl), 172.3(carbonyl), 172.2(carbonyl), 172.2(carbonyl), 171.4(carbonyl), 170.7(carbonyl), 168.4(carbonyl), 157.5 (imine), 157.5 (imine), 62.1 (Ser-β), 61.8 (Ser-β), 57.4 (Ile-α), 56.9 (Lys-α), 56.2 (Leu-α), 53.4, 53.3, 52.8, 52.5, 52.1 (Leu-α), 50.5 (Asn-α), 40.9 (Arg-δ), 40.9 (Arg-δ), 40.7, 40.2, 39.4 (Lys-ε), 36.8 (Ile-β), 36.7 (Asn-β), 31.6, 29.1, 28.6, 26.9, 25.5, 24.6, 24.5, 24.4 (Ile-γ), 23.0 (Leu-δ), 22.9 (Leu-δ), 22.6 (Leu-β), 21.2 (Leu-δ), 21.1 (Leu-δ), 14.5 (Ile-γ′), 10.1 (Ile-6). HRMS (ESI/Q-ToF): HRMS (ESI/Q-ToF): calcd for C₄₆H₈₈N₁₈O₁₂, 1084.6829; found 1084.6833.

PA Purity Measurements with LC-MS

LC-MS was performed on an Agilent 1200 system with an Agilent 6520 Q-TOF detector. All gradient methods followed: acetonitrile at 5% for 5 min at 50 μl/min, then 5-95% over 25 min at 50 μl/min followed by 95% for 5 min at 50 μl/min. Ammonium hydroxide (0.1% v/v) or formic acid (0.1% v/v) was added to all basic or acidic solvents respectively. For acidic methods a Phenomenex Jupiter C-12; 100×1 mm, 5 μm was used. For basic methods, Phenomenex Gemini C-18, 100×1 mm; 5 μm was used.

### Accurate Mass Analysis

High resolution mass spectra (HRMS) were recorded on an Agilent 6210A LC-TOF mass spectrometer in positive ion mode using electrospray ionization.

### PA Preparation

The samples were prepared by dissolving 10 mg of lyophilized peptide powders (E2 PA, GDNF PA and Scr PA) in a 125 mM NaCl and 3 mM KCl solution to a PA concentration of 1 wt %.

The co-assemblies were prepared by mixing the PA solutions in the volume ratios described in table 2, then the solutions were vortexed. All solutions were adjusted to pH of 7.4 by the addition of 1 M NaOH. The sample solutions were further annealed at 80° C. for 30 min in a water bath, then slowly cooled to ambient temperature overnight.

### Cryogenic Transmission Electron Microscopy (Cryo-TEM)

Samples for cryo-TEM were dissolved and annealed according to the above-described procedure and held at room temperature until plunge-freezing. 300-mesh copper grids with lacey carbon support (EMS) were glow discharged for 15 seconds using a PELCO easiGLow Glow Discharge Cleaning System (Ted Pella) and used immediately. Samples were diluted 10-fold in milliQ water and vortexed briefly immediately before 7 μL of sample solution was deposited on a copper grid inside a Vitrobot Mark IV (FEI) vitrification robot at room temperature with 95-100% humidity. The grids were blotted to wick away excess sample and plunge frozen into liquid ethane. Samples were transferred into a liquid nitrogen bath for holding until imaging, where they were placed into a Gatan 626 cryo-holder through a cryo-transfer stage. Cryo-TEM was performed using a JEOL 1230 TEM working at 100 kV accelerating voltage. Images were acquired using a Gatan 831 CCD camera.

### Small Angle X-Ray Scattering (SAXS), Mid-Angle X-ray Scattering (MAXS), Wide Angle X-Ray Scattering (WAXS)

Experiments were performed at beamline 5-ID-D of the DuPont-Northwestern-Dow Collaborative Access Team (DND-CAT) Synchrotron Research Center at the Advanced Photon Source, Argonne National Laboratory. PA samples were prepared at 2 wt % irradiated for 2 seconds. Data was collected with an X-Ray energy at 17 keV (1=0.83 Å). Sample to detector distances were as follows: 201.25 mm for SAXS, 1014.2 mm for MAXS, and 8508.4 mm for WAXS. The scattering intensity was recorded in the interval 0.002390<q<4.4578 Å⁻¹. The wave vector q is defined as =(4π/λ) sin(θ/2), where θ is the scattering angle. Azimuthal integration (Fit2D) was used to average 2D scattering images to produce 1D profiles of intensity versus q. Background scattering patterns were obtained from samples containing 125 mM NaCl and 3 mM KCl and subtracted from experimental data. All data was analyzed using the Irena software package running on IgorPro software.

### Fourier Transformed Infrared (FT-IR) Spectroscopy

FT-IR spectra were recorded on a Nexus 870 spectrometer (Thermo Nicolet) FT-IR spectrometer. Samples were prepared in deuterated water (D₂O) and placed between two CaF₂ windows with a spacing of 50 μm. The final spectra result from 32 scans with 1 cm⁻¹ resolution and atmospheric CO₂ and H₂O were background subtracted.

### Atomic Force Microscopy (AFM)

PA solutions were prepared as described in the main text. Sample solutions were diluted 10× in 150 mM NaCl and deposited on freshly cleaved mica surfaces for ~1 min, and the excess solution was rinsed with 150 mM NaCl. The samples were then rinsed with 150 mM NaCl 20 mM CaCl₂ to immobilize the nanostructures on the mica surface, and measurements were performed in the liquid environment. AFM images were captured in PeakForce tapping mode on a Dimension Icon AFM (Bruker) with a silicon nitride cantilever (SNL10-A, Bruker) in a liquid cell. Images were flattened to correct sample tilt before analysis.

### Circular Dichroism (CD) Spectroscopy

Each PA sample was diluted to concentrations between 0.01-0.04 wt % in either H₂O (no salt samples) or buffer containing 150 mM NaCl and 3 mM KCl (high salt). CD spectra was recorded on a JASCO model J-815 spectropolarimeter using a quartz cell of 0.5 mm optical path length. Continuous scanning mode was used with a scanning speed of 100 nm per minute with the sensitivity set to standard mode. High tension (HT) voltage was recorded for each sample to ensure that the measurement was not saturated. An accumulation of three measurements was used and a buffer sample was background-subtracted to obtain final spectra. The final spectra were normalized to final concentration of each sample using a molar averaged molecular weight.

### Scanning Electron Microscopy (SEM)

All samples were fixed in a 2.5% glutaraldehyde (GTA) 4% paraformaldehyde (PFA) phosphate buffered saline (PBS) solution for at least 20 min. They were dehydrated in a series of ethanol solutions increasing in concentration from 30-100% with ten-minute incubations for each incremental increase. Ethanol was then exchanged and removed with critical point drying using a Tousimis Samdri-795. Dehydrated samples were mounted using a carbon glue tape on sample studs and stored under vacuum until the day of imaging. Immediately prior to imaging, samples were coated with 16 nm of osmium (Filgen, OPC-60A) to create a conductive surface. Images were taken using a Hitachi SU8030 instrument using an accelerating voltage of 2-3 kV.

### Rheological Measurements

An Anton Paar MCR302 Rheometer with a 25 mm cone plate was used for all rheological studies. 150 μL of PA liquid was placed on the sample stage and 30 μl of 150 mM CaCl₂) (final concentration 25 mM) was placed on the plate while in the instrument loading position. The instrument was set to 37° C. to simulate in vitro and in vivo conditions. The plunger was lowered to the measuring position and a humidity collar was added to prevent sample evaporation during the measurement. The sample was equilibrated for 30 minutes with a constant angular frequency of 10 rad/s and 0.1% strain. The angular frequency was then decreased from 100 rad/s to 1 rad/s over 21 points and the storage and loss modulus were recorded. The % strain was increased from 0.1 to 100% over 31 points and the storage and loss moduli were recorded.

### Cell Culture

iPSC-derived human midbrain floorplate dopaminergic neurons (iCell® DopaNeurons, Cellular Dynamics) were used. The cells were thawed following manufacturer's instructions. Briefly, the cells were seeded on coverslips coated with PLO (0.01%) and 3.3 μg/ml laminin solution at 4.0×10⁴. The recommended by Cellular Dynamics was used following the instructions provided by the manufacturer. The preparation consist of the addition of two supplements to a basal media (for the starvation condition in WB experiments only the basal media without the addition of supplements was used). The media was changed the next day of seeding, and then media was changed every 3 days. The following soluble treatments were added: GDNF protein (Stemcell Technologies) 10 or 100 (3.3 nM) ng/m, and mimetic peptide (ILKNLSRSR (SEQ ID NO: 1) 100 ng/ml or 3.3 nM. The GDNF or peptide was added every time media was changed.

### PA Treatments

### PA Gels on the Top of Cells:

Cell media was removed and 30 μl of PA was added with a P200 pipette. Care was taken to avoid the contact between the tip and the cells. The PA was homogeneously distributed around the coverslip. Immediately, fresh cell media was added very slowly.

### PA Coatings:

Coverslips coated with poly-D-lysine (PDL) 0.1 mg/ml (Sigma-Aldrich) were used. Coverslips were washed once with sterile water and allowed to dry in air inside a flow hood. 15 μl of PA was deposited on a piece of sterile parafilm. The coverslips were placed in contact with the PA for 1 hour. Then, coverslips were placed in a 24-well plate with the side previously in contact with the PA facing upwards. Gelling solution comprised of 125 mM NaCl, 3 mM KCl, and 25 mM CaCl₂ was added for 1 min. Gelling solution was removed, and coverslips were washed with sterile PBS 1×. PBS was withdrawn and cell media was added.

### PA Scaffolds:

iCell® DopaNeurons (Cellular Dynamics) were thawed according to manufacturer instructions to obtain 1.0×10⁵ in 10 μl of cell media. Cell suspension was mixed with 20 μl of 1 wt % annealed PA of 15 vol % Scr PA co-assembly or 15 vol % GDNF PA co-assembly by pipetting. This mixed solution (30 μl) was placed in a coverslip and let it rest for 5 min inside the incubator. Then, 500 μl of cell media was added carefully to avoid disrupt the gel.

### Cell Viability

Cell media with Calcein-AM 1 μM and propidium iodide 2.5 μM (Invitrogen) was added and placed in the incubator for 20 min according to manufacturer's instructions. The solution was removed, and the samples were washed with PBS 1×. Coverslips were mounted, and the confocal images were acquired immediately. Experiments were performed by triplicate.

### PA Internalization Assay

TAMRA-GDNF PA was prepared by mixing 85 vol % of E2 PA (1 wt %) (no dye) with 15 vol % of C16-VVAAEEK (SEQ ID NO:16)-TAMRA-ILKNLSRSR (SEQ ID NO: 1) (1 wt %). The cells were seeded on 24 well plate with glass-like polymer bottom (Cellvis, cat. No. P24-1.5P) at a density of 4.0×10⁴. Cell culture conditions are described above. The neurons were incubated with 10 μg/mL cholera toxin B-AlexaFluor647 (Invitrogen, cat. No. C34778) at 37° C. for 1 hour. The neurons were treated with TAMRA-GDNF PA (10.73 mM of PA concentration) for either 10 minutes or 1 hour at 37° C. prior to being washed and stored in warmed media. Imaging was performed using a Nikon AXR confocal microscope in a live cell chamber at 37° C. under 5% CO₂. For clearer visualization of PA internalization, Imaris Bitplane software (Oxford Instruments) version 9.2.1 was used. Briefly, the Surface function was used, and the threshold was adjusted to match the fluorescence signal of the images obtained with the Nikon AXR confocal. To distinguish PA from the neuron, separate surfaces (Imaris function) were employed for each and subsequently images were superimposed. Then, the neuron membrane staining surfaces were adjusted to 70% transparency to visualize GDNF PA inside the neurons.

### Western Blot

For receptor activation and downstream signaling experiments 850,000 cells were seeded in 6 well plates precoated with poly-D-lysine (Sigma Aldrich) until they reach about 70% of confluency. Treatments were added for 10 min and then the protein was harvested. For the GDNF native protein treatment, we added 100 ng/ml of human recombinant GDNF (Stemcell Technologies) in media, trophic factor was added each time the media was changed(every 4 days). For neuroprotective experiments with 6-OHDA (Sigma Aldrich) the cells were seeded on PA coatings except for Starvation (strv) and PDL conditions in which Poly-D-lysine coated wells were used. The neurotoxin 6-OHDA (100 μM) was added for 1 hour and then the protein was extracted. The 25 μg protein was separated by 4-20% SDS-polyacrylamide gel electrophoresis and electrotransferred to a nitrocellulose membrane (Bio-Rad). Membranes were blocked with 5% milk solution (Bio-Rad) for 1 hour and then primary antibodies were added for an overnight incubation at 4° C. The following primary antibodies were used: rabbit anti-Actin (1:2000, Sigma Aldrich), rabbit anti-Fyn (1:1000, Cell Signaling), anti-pSrc (1:1000, Cell Signaling), rabbit anti-ERK/2 (1:1000, Cell Signaling), rabbit anti-pERK 1/2 (1:1000, Cell Signaling), rabbit anti-RET (1:1000, Cell Signaling), rabbit anti-pRET (1:500, Cell Signaling), rabbit anti-TH (1:1000, Pel-freez) and mouse anti-Tuj-1 (1:5000, BioLegend), GAPDH (1:2000, Millipore). Subsequently, the membranes were incubated with secondary antibodies (1:1000; ThermoFisher). Densitometry analysis, normalized to actin or total protein (ERK or RET) as a control for protein loading, was accomplished using ImageJ software (National Institutes of Health, USA). The experiments were performed in triplicate.

### Pharmacological Inhibition of RET and NCAM

The iPSC-derived human dopaminergic neurons were seeded. For RET receptor inhibition, cells (2 DIV) were incubated for 24 hours with Vandetanib (Tocris) (1 μM). In a simultaneous experiment, the neurons were incubated with PP2 (Tocris) (1 μM) for 1 hour to inhibit Fyn (NCAM downstream signaling protein) (experiments by triplicate, n=200 cells). Then, GDNF protein (100 ng/ml) or 15 vol % GDNF PA (gel on the top) was added for two days. Cells were assessed by a cell viability assay, fixed for TH immunostaining, or used for protein harvesting.

Human Embryonic Stem Cell Maintenance and hMLOs Generation

This study has been approved by Northwestern University Committee on Human Stem Cell Research. Human embryonic stem cell (hESC) line, HUES64 (passage 40) was maintained on matrigel-coated dishes with mTeSR1 or mTeSR plus (Stem Cell Technologies). The cells were dissociated to single cells with 1 mM EDTA or Accumax (Innovative Cell Technologies) for passaging or differentiation, respectively. Matrigel lots have been routinely tested to optimize stem cell maintenance. ROCK inhibitor was used to prevent cell death upon cell dissociation. hMLOs were generated. Briefly, 10,000 cells were plated on V-bottomed conical wells (Sumitomo Bakelite) to form the embryonic bodies. Cells were maintained with induction media (DMEM/F12:Neurobasal, 1:1), N2 supplement 1:100 (Invitrogen), B27 without vitamin A1:50 (Invitrogen), 1% Glutamax (Invitrogen), 1% minimum essential media-nonessential amino acid (Invitrogen), and 0.1% β-mercaptoethanol (Invitrogen) with 1 μg/ml heparin (Sigma-Aldrich), 10 μM SB431542 (Stemgent), 200 ng/ml Noggin (Prospec), 0.8 μM CHIR99021 (Reagents Direct), and 10 μM ROCK inhibitor Y27632 (Calbiochem). At day 4, 100 ng/ml SHH-C2511 (R&D Systems) and 100 ng/ml FGF8 (R&D Systems) was added. After 7 DIV, the media was removed and 30 μl of reduced growth factor Matrigel was added, followed by incubating the cells at 37° C. for 30 min. Cells were maintained with in Neurobasal medium, N2 supplement 1:100 (Invitrogen), B27 without vitamin A 1:50 (Invitrogen), 1% GlutaMAX (Invitrogen), 1% minimum essential media-nonessential amino acid (Invitrogen), and 0.1% β-mercaptoethanol (Invitrogen) supplemented with 2.5 μg/ml insulin (Sigma-Aldrich), 200 ng/ml laminin (Sigma-Aldrich), 100 ng/ml SHH-C2511 (R&D Systems), and 100 ng/ml FGF8 (R&D Systems) for 24 hours. The organoids were transferred to low-attachment 6 well-plates (Costar). Differentiation media containing Neurobasal medium, N2 supplement 1:100 (Invitrogen), B27 without vitamin A 1:50 (Invitrogen), 1% GlutaMAX (Invitrogen), 1% minimum essential media-nonessential amino acid (Invitrogen), 0.1% β-mercaptoethanol (Invitrogen), 10 ng/ml BDNF (Peprotech), 10 ng/ml GDNF (Peprotech), 100 μM ascorbic acid (Sigma-Aldrich), and 125 μM db-cAMP (Sigma-Aldrich). Penicilin (100 U/ml) and streptomycin (100 μg/ml) were added. Organoids were cultured in an orbital shaker to promote the nutrient and gas exchange.

### Organoid Embedding Process

At 80 DIV a high population of TH-positive cells was observed, therefore organoids at this stage were used to evaluate the bioactivity of PAs. Organoids were transferred from the 6 well plates to Lab Tek® 4 well-chambers slide (one organoid per chamber). The organoids were moved carefully to the corner of the chamber with a sterile tip, then the media was removed and enough PA solution to cover the organoid was added (50 μl) (the PA solution was prepared as previous experiments, 15 vol % of Scr PA and GDNF PA). Immediately, gelling solution (25 μl) was added, after 3 minutes media was added. Media was changed every 3 days. After one week, organoids were fixed with 4% PFA for two hours. The experiments were performed in triplicate. In total, 9 organoids per condition were generated.

### Immunostaining and Imaging

All samples were fixed with PFA 4%, cells seeded on coverslips were fixed for 15 minutes at RT, cells embedded of PA gels were fixed for one hour at RT, organoids embedded on PA were fixed for 2 hours at RT and organoids without PA were fixed overnight at 4° C. After fixation organoids without PA were transferred in a 30% sucrose solution in PBS overnight at 4° C., then the organoids were embedded in a O.C.T. solution (Sakura Finetek) and maintain at −80° C. until cryosectioning. The organoids were sliced at a thickness of 10 μm with a Leica CM1520 cryostat. Cells on coverslips and embedded in gels, and slices of organoids were permeabilized with 0.3% Triton 100× and 10% Normal Goat Serum in PBS for 1 hour, organoids embedded in PA were permeabilized for 2 hours. Samples were incubated with primary antibodies in PBS with 10% Normal Goat Serum overnight and with secondary antibodies in PBS with 10% Normal Goat Serum for 2 hours at RT. After antibodies incubations samples were washed with PBS/Bovine Serum Albumin 0.1% 3× for 5 min for cells on coverslips or 4× for 15 min for cells and organoids embedded in PA gels. The following primary antibodies were used: rabbit anti-TH (Tyrosine Hydroxylase marker 1:1000, Pel-freez), mouse anti-TH (1:1000, RD Systems), mouse anti-MAP2 (marker for mature neurons 1:1000, Thermo Fisher), mouse anti Tuj1 (1:1000, Biolegend), mouse anti-PSD95 (Post-synaptic marker 1:1000, Thermo Fisher), mouse anti-Synaptophysin (Pre-synaptic marker 1:100, Cell Signaling), rabbit anti-Cleaved Caspase-3 (marker for apoptosis 1:500, Cell Signaling), goat anti-Girk2 (midbrain dopaminergic marker 1:200, abcam), mouse anti-nestin (neuronal precursor marker 1:500, Novus Biologicals) mouse anti-Tuj1 (neuronal marker 1:1000, Biolegend), and mouse anti-DAT (dopamine transporter marker 1:200, Atlas antibodies). The following secondary antibodies were used: Alexa 488 or Alexa 568 (1:1000 Thermo Fisher). Stained cells in PA coating were visualized using a Nikon AIC confocal microscope. Cells embedded in PA gels were examined with a Nikon A1R-MP multiphoton microscope (we transferred the coverslips with the PA gels from the 24-well plate to a 35 mm dish for imaging). Organoids images were taken on a Nikon AXR confocal. We used a Zeiss AxioZoom V16 Stereo Microscope to visualize the organoid embedded on the PA. The imaging conditions were identical in all groups in every experiment.

### Imaging and Morphometric Analysis

Confocal images of PA coatings were reconstructed using NIS Elements Advanced Research Microscope Imaging. Images were processed using ImageJ/Fiji (National Institutes of Health) software. Number of dendrites, Sholl analysis and colocalization were analyzed using Fiji. Axonal length was measured using NeuronJ plugin in ImageJ. The experiments were performed by triplicate (in total 200 cells per group). Sholl analysis quantified the number of intersections that the neurites have with concentric circles from the cell soma, for this purpose we used circles with a 40 μm of distance between them for cell coating experiments and 10 μm for scaffold experiments. Experiments were performed by triplicate, n=200 cells per condition. 3D images from multiphoton microscopy of PA gels samples were reconstructed and analyzed using Imaris software (version 8.1, Bitplane Scientific software). Volume of fluorescence signal (TH and MAP2) was analyze using Voxel Counter plugin of ImageJ. For morphology, quantification a minimum of 30 randomly selected neurons were analyzed per condition of at least two independent experiments (in total 60 cells per group). To evaluate the axonal extension from organoids the confocal images were converted to 8-bit, then to remove the fluorescence noise from the PA it was used a Tubeness plugin in Fiji software using a 1-sigma value. To obtain a 3D digital reconstruction of the axons and measure the length we used the Simple Neurite Tracer plugin. Volume projection image with the depth scale of 3D-cultres and organoids were generated using the volume projection function of NIS Elements software from Nikon. Adobe Photoshop CC software version 20.0.7 was used to adjust brightness and contrast, and to prepare the figures.

### Electrophysiology

Spontaneous pacemaking activity was recorded on iCell® DopaNeurons (Cellular Dynamics) cultured by 30-35 DIV. Cultures were transferred to a recording chamber on a fixed-stage inverted microscope (Diaphot 200; Nikon). Experiments were performed at 32° C. in neurons seeded on Scr PA 15 vol %, GDNF PA 15 vol % and PDL coatings with GDNF (100 ng/ml). The recording chamber was perfused (1-2 ml/min) with HEPES-based solution (in mM): 140 NaCl, 5 KCl, 1 MgCl₂, 2 CaCl₂), 10 HEPES, 10 glucose, 10 sucrose; pH 7.4, osmolarity 300-305 mOsm/L. Patch pipettes (3-5 MW) were filled with internal solution containing the following (in mM): 135 K-MeSO₄, 5 KCl, 5 HEPES, 0.05 EGTA, 10 phosphocreatine-di(tris), 2 ATP-Mg, 0.5 GTP-Na, pH: 7.25-7.30, osmolarity 285-295 Osm/L. Recording patch pipettes were prepared with a horizontal puller (model P-97; Sutter Instruments) using borosilicate glass with filament (outer diameter 1.5 mm, inner diameter 0.86 mm). Somatic cell-attached voltage-clamp recordings were obtained with a Multi-Clamp 700B amplifier (Molecular Devices) interfaced to a Pentium-based PC running pClamp 10.6 (Molecular Devices). The signals were filtered at 1 kHz and digitized at 10 kHz with a Digidata 1440A (Molecular Devices). Analysis of instantaneous firing frequency was done in Clampfit 10.6 (Molecular Devices). The experiments were performed by triplicate. Dopaminergic neurons were identified based on their characteristic slow, autonomous pacemaking (3.8 Hz)¹⁰⁷.

### RNA Sequencing Libraries

RNA quality was determined with the Agilent Bioanalyzer 2100, accepting RNA integrity numbers (RIN) of >7 and quantified using Qubit. Directional mRNA libraries were prepared using Illumina TruSeq mRNA Sample Preparation Kits per manufacturer's instructions. Briefly, polyadenylated mRNAs were captured from total RNA using oligo-dT selection. Next, samples were converted to cDNA by reverse transcription, and each sample was ligated to Illumina sequencing adapters containing unique barcode sequences. Barcoded samples were then amplified by PCR and the resulting cDNA libraries by quantified using qPCR. Finally, equimolar concentrations of each cDNA library were pooled and sequenced on the Illumina HiSeq 4000.

### RNA Sequencing Analysis

The quality of reads, in FASTQ format, was evaluated using FastQC. Reads were trimmed to remove Illumina adapters from the 3′ ends using cutadapt¹⁰⁸. Trimmed reads were aligned to the Homo sapiens genome (mm10) using STAR¹⁰⁹. Read counts for each gene were calculated using htseq-count¹¹⁰ in conjunction with a gene annotation file for mm10 obtained from Ensembl (useast.ensembl.org). Normalization and differential expression were calculated using DESeq2 that employs the Wald test¹¹¹. The cutoff for determining significantly DEGs was Fold change ±1, P-value <0.05. Origin2023b software was used to plot the DEGs in the volcano plot.

### Gene Ontology Analysis

To perform the gene ontology, protein coding genes with ±1 fold change values were used. The SynG02020⁷⁰ library was used to evaluate the DEGs in a synaptic context. The top 10 upregulated and top 10 downregulated enriched terms were evaluated. To identify the most significant molecular function (MF) GO terms of the upregulated protein-coding genes, the g:Profiler web⁷² was used. The functional profiling option was selected under the following parameters: Organism-Homo sapiens, customed-background list, Significance threshold-g:SCS threshold, User threshold-0.05, Numeric IDs treated as-entrezgene_acc. The Venn diagram function from (https://www.biotools.fr) was used to compare the genes with ≥1-fold change values.

### Statistical Analysis

Statistical analysis was performed using the software GraphPad Prism version 6.01. Data from multiple group experiments were analyzed by One-way ANOVA followed by Tukey post hoc test. Comparison data from two experimental groups were analyzed by T-test. Error bars indicate the standard error of the mean. Differences were considered statistically significant when P<0.05, and the significance level is shown by * P<0.05; * * P<0.01; * * * P<0.001; * * * * P<0.0001.

### Data Availability

The RNA-Seq Raw data have been deposited in the NCBI's Gene Expression Omnibus and can be access through GEO number (GSE253788).

1. A GDNF mimetic peptide amphiphile comprising a hydrophobic tail, a structural peptide segment, a charged peptide segment, and a glial derived neurotrophic factor (GDNF) mimetic peptide.

2. The GDNF mimetic peptide amphiphile of claim 1, wherein the GDNF mimetic peptide comprises a sequence having at least 75% identity with ILKNLSRSR (SEQ ID NO: 1).

3. The GDNF mimetic peptide amphiphile of claim 1, wherein the GDNF mimetic peptide comprises a sequence having at least 88% identity with SEQ ID NO: 1.

4. The GDNF mimetic peptide amphiphile of claim 1, wherein the GDNF mimetic peptide comprises SEQ ID NO: 1.

5. The GDNF mimetic peptide amphiphile of claim 1, wherein the hydrophobic tail comprises an 8-24 carbon alkyl chain (C₈₋₂₄), the structural peptide segment has propensity for forming n-sheet conformations, the charged peptide segment comprises an acidic, basic, or zwitterionic peptide segment, and/or the GDNF mimetic peptide is attached to the charged peptide segment by a linker.

6. The GDNF mimetic peptide amphiphile of claim 1, wherein the structural peptide segment comprises 2 to 8 non-polar residues.

7. The GDNF mimetic peptide amphiphile of claim 6, wherein the structural peptide segment comprises VVAA (SEQ ID NO: 2).

8. The GDNF mimetic peptide amphiphile of claim 1, wherein the charged peptide segment comprises EE, EEE, EEEE (SEQ ID NO: 4), KK, KKK, or KKKK (SEQ ID NO: 9).

9. The GDNF mimetic peptide amphiphile of claim 1, wherein the peptide amphiphile comprises C₈₋₂₄-VVAAEEILKNLSRSR (SEQ ID NO: 17).

10. A nanofiber comprising the GDNF mimetic peptide amphiphile of claim 1.

11. The nanofiber of claim 10, further comprising one or more filler peptide amphiphiles, wherein the filler peptide amphiphiles comprise a hydrophobic tail, a structural peptide segment, and a charged peptide segment, and wherein the filler peptide amphiphiles do not comprise the GDNF mimetic peptide.

12. The nanofiber of claim 11, wherein the filler peptide amphiphiles do not comprise a bioactive segment.

13. The nanofiber of claim 11, comprising:
a) about 5-50% GDNF mimetic peptide amphiphiles and about 50-95% filler peptide amphiphiles;
b) about 12% to about 28% GDNF mimetic peptide amphiphiles and about 72% to about 88% filler peptide amphiphiles; or
c) about 15% GDNF mimetic peptide amphiphiles and about 85% filler peptide amphiphiles.

14. A composition comprising the GDNF mimetic peptide amphiphile of claim 1.

15. A method of treating a nervous system injury or disease in a subject, comprising administering the composition of claim 14 to the subject.

16. The method of claim 15, wherein the composition is administered to the subject concurrently with cell replacement therapy.

17. A scaffold comprising the nanofiber of claim 10.

18. A cell or an organoid cultured on the scaffold of claim 17.

19. A system comprising the scaffold of claim 17 and a cell or an organoid cultured on the scaffold.

20. A method of treating Parkinson's disease in a subject, the method comprising administering to the subject the system of claim 19.
